(19) United States
(12) Patent Application Publication
(10) Pub. No.: US 2022/0372071 A1
(43) Pub. Date: Nov. 24, 2022

(54) HIGH SALT WASHES DURING CATION EXCHANGE CHROMATOGRAPHY TO REMOVE PRODUCT-RELATED IMPURITIES

(71) Applicant: AMGEN INC., Thousand Oaks, CA (US)

(72) Inventors: Malhar R. AMBHAIKAR, Thousand Oaks, CA (US); Luis DIAZ, Thousand Oaks, CA (US); Natalia R. GOMEZ, Playa Vista, CA (US); Benjamin J. TILLOTSON, Bainbridge Island, WA (US)

(73) Assignee: AMGEN INC., Thousand Oaks, CA (US)

(21) Appl. No.: 17/773,892

(22) PCT Filed: Nov. 4, 2020
(86) PCT No.: PCT/US2020/058774

§ 371 Date: May 3, 2022

Related U.S. Application Data
(60) Provisional application No. 62/931,874, filed on Nov. 7, 2019.

(51) Int. Cl. C07K 1/18; C07K 16/46; B01D 15/36; B01D 15/42; B01D 15/38; B01D 61/14

(52) U.S. Cl. CPC C07K 1/18; C07K 16/468; B01D 15/362; B01D 15/426; B01D 15/3809; B01D 61/145; C07K 2317/31; B01D 2315/16

(57) ABSTRACT

The invention relates to methods for removal of low isoelectric point product-related impurities during cation exchange purification operations.

This application claims the benefit of U.S. Provisional Application No. 62/931,874, filed Nov. 7, 2019, which is hereby incorporated by reference.

### FIELD OF DISCLOSURE

The present invention relates to the field of biopharmaceutical manufacturing. In particular, the invention relates to methods for removal of product-related impurities of multi-specific proteins cation exchange purification operations.

### BACKGROUND

Antibody products are the largest sector of the biopharmaceuticals market and could easily reach hundreds of billions in sales over the next decade. The commercial development of therapeutic antibodies began in the 1980s with the approval of the first therapeutic monoclonal antibody and has continued to evolve and expand ever since. While monoclonal antibodies are able to bind to a target with high affinity and specificity, and have been very successful for treating some indications, they also have limitations as therapeutics. Monoclonal antibodies can only bind to a single target; however, many diseases are multifactorial. In cancer immunotherapy, a treatment aimed at a single target may not be sufficient to completely destroy or immobilize cancer cells. In addition, some patients receiving monoclonal antibody therapies may fail to respond to treatment or even develop drug resistance after a time.

New antibody-like structures such as antibody Fab fragments, Fc-fusion proteins, antibody-drug conjugates, glycol-engineered antibodies, and most especially, bispecific and other multispecific antibody-like structures have been developed to meet these challenges. These antibody-like structures, particularly bispecific antibodies, offer improvements over traditional monoclonal antibody therapeutics, such as multi-target affinity, and are proving to be effective next-generation of biotherapeutics with an enormous variety of formats that can be developed to meet even more challenging therapeutic indications.

Bispecific antibodies are the most diverse group of these antibody-like structures with an ever-increasing variety of frameworks to meet the challenges an even broader scope of therapeutic indications. These structures combine the binding properties of antibodies with additional molecular properties engineered into the frameworks to suit needs of the targeted disease indications. Bispecific antibodies are being developed for a variety of indication and uses, such as redirecting immune effector cells to tumor cells for immune response against cancer, blocking signaling pathways, targeting tumor angiogenesis, blocking cytokines, crossing the blood-brain barrier, diagnostic assays, treatment of pathogens, and as delivery agents. (Sedykh et al., Drug Design, Development and Therapy 18(12), 195-208, 2018; Walsh, Nature Biotechnology, 32(10), 992-1000, 2014; Ecker et al., mAbs 7(1), 9-14, 2015; Spiess et al., Mol Immunol 67, 95-106, 2015; Fan et al., J Hematol & Oncology 8:130-143, 2015; Williams et al., Process Design for Bispecific Antibodies, Biopharmaceutical Processing, Development, Design and Implementation of Processes, Jagschies et al., editors, Elsevier Ltd, pages 837-855, 2018).

Development of these multispecific proteins brings new biomanufacturing challenges, particularly with regard to product instability and low expression yields. In particular, purification of multispecific proteins is complicated by the formation of product-related variants, such as homodimers, half-antibodies, aggregates, high and low molecular weight species and the like. These product-related variants share similar structural and physical properties, such as charge, with the multispecific protein of interest, making them difficult to separate from during purification. These product-related impurities lower the yield and activity of the multispecific drug product.

Product-related impurities having similar charge (isoelectric point, pI) to a multispecific protein of interest may co-elute with the multispecific protein during cation exchange chromatography unit operations, complicating purification and lowering yield. It would be beneficial to separate the low pI product-related impurities prior to elution. The invention described herein meets this need by providing high salt wash conditions for removal of these low pI product-related impurities during cation exchange chromatography.

### BRIEF SUMMARY OF THE INVENTION

The invention provides a method for purifying a multispecific protein comprising loading a sample comprising a multispecific protein onto a cation exchange chromatography medium; washing the cation exchange medium with at least one wash buffer comprising 100-147 mM sodium chloride; and eluting the multispecific protein from the cation exchange chromatography resin. In one embodiment, at least one wash buffer comprises 100-125 mM sodium chloride. In one embodiment at least one wash buffer comprises 100-105 mM sodium chloride. In one embodiment at least one wash buffer comprises 105-147 mM sodium chloride. In one embodiment at least one wash buffer comprises 105-125 mM sodium chloride. In one embodiment at least one wash buffer comprises 125-147 mM sodium chloride. In one embodiment at least one wash buffer comprises acetate. In a related embodiment at least one wash buffer comprises acetate, pH 5.0±0.05% to pH 5.0±0.1%. In a related embodiment at least one wash buffer comprises acetate, pH 4.91-5.1. In a related embodiment at least one wash buffer comprises acetate, pH 4.9, 5.0, or 5.1. In another related embodiment the wash buffer comprises 100 mM acetate. In one embodiment at least one wash buffer comprises acetate, 100-125 mM sodium chloride. In one embodiment at least one wash buffer comprises acetate, 100-105 mM sodium chloride. In one embodiment at least one wash buffer comprises acetate, 105-147 mM sodium chloride. In one embodiment at least one wash buffer comprises acetate, 105-125 mM sodium chloride. In one embodiment at least one wash buffer comprises acetate, 125-147 mM sodium chloride.

In one embodiment the cation exchange medium is washed with at least two wash buffers. In one embodiment, the cation exchange medium is washed with at least three wash buffers.

In one embodiment the cation exchange medium is washed with at least two wash buffers, at least one of the wash buffers comprising 0-147 mM sodium chloride. In a related embodiment the cation exchange medium is washed with at least two wash buffers, at least one of the wash buffers comprising 0-70 mM sodium chloride. In one embodiment the cation exchange medium is washed with at least two wash buffers, at least one wash buffer comprising acetate, 0 mM sodium chloride, followed by a wash buffer comprising acetate, 100-147 mM sodium chloride. In a related embodiment the cation exchange medium is washed with a wash buffer comprising acetate, 0 mM sodium chloride, followed by a wash buffer selected from the group consisting of a wash buffer comprising acetate, 100 mM sodium chloride, a wash buffer comprising acetate, 105 mM sodium chloride, or a wash buffer comprising acetate, 125 mM sodium chloride. In one embodiment the cation exchange medium is washed with a wash buffer comprising acetate, 100-147 mM sodium chloride, followed by a wash buffer comprising acetate, 0-70 mM sodium chloride. In a related embodiment the cation exchange medium is washed with a wash buffer comprising acetate, 100-147 mM sodium chloride, followed by a wash buffer comprising acetate, 0 mM sodium chloride. In another related embodiment the cation exchange medium is washed a wash buffer comprising acetate, 100-147 mM sodium chloride, followed by a wash buffer comprising 70 mM sodium chloride.

In one embodiment the cation exchange medium is washed with at least three wash buffers, a first wash buffer comprising acetate, 0 mM sodium chloride, followed by a second wash buffer comprising acetate, 100-147 mM sodium chloride, followed by a third wash buffer comprising acetate, 0 mM sodium chloride or a wash buffer comprising acetate, 70 mM sodium chloride. In a related embodiment the cation exchange medium is washed with a first wash buffer comprising acetate, 0 mM sodium chloride, followed by a second wash buffer selected from the group consisting of a wash buffer comprising acetate, 100 mM sodium chloride, a wash buffer comprising acetate, 105 mM sodium chloride, followed by a wash buffer comprising acetate, or a wash buffer comprising acetate, 125 mM sodium chloride, followed by a third wash buffer comprising acetate, 0 mM sodium chloride. In a related embodiment the cation exchange medium is washed with a first wash buffer comprising acetate, 0 mM sodium chloride, followed by a second wash buffer comprising acetate, 147 mM sodium chloride, followed by a wash buffer comprising acetate, followed by a third wash buffer comprising acetate, 70 mM sodium chloride.

In one embodiment the cation exchange medium is washed with 2.5 mM/CV of a wash buffer comprising 147 mM sodium chloride.

In one embodiment the multispecific protein is eluted from the cation exchange medium by a gradient. In a related embodiment the gradient is a linear or step gradient. In a related embodiment the gradient is a salt gradient. In a related embodiment the buffers used to form the elution gradient comprises 0-1M sodium chloride. In another related embodiment at least one of the buffers used to form the elution gradient comprises 70-500 mM sodium chloride. In another related embodiment at least one of the buffers used to form the elution gradient comprises 125 mM sodium chloride. In a related embodiment at least one wash buffer and one elution buffer comprise 125 mM sodium chloride.

In one embodiment the cation exchange medium is loaded with at least 10 g/L of the multispecific protein. In one embodiment the cation exchange medium is loaded with 10 g/L to 40 g/L of the multispecific protein. In a related embodiment the cation exchange medium is loaded with 15 g/L to 30 g/L. In a related embodiment the cation exchange medium is loaded with 25 g/L-40 g/L. In one embodiment the cation exchange medium is loaded with 10 g/L of the multispecific protein, washed with a wash buffer comprising 105 mM sodium chloride and eluted in a salt gradient at 8 mM/CV. In one embodiment the cation exchange medium is loaded with 15 g/L to 30 g/L of the multispecific protein, washed with a wash buffer comprising 147 mM sodium chloride. In one embodiment the cation exchange medium is loaded with 25 g/L to 40 g/L of the multispecific protein, wherein at least one wash buffer and one elution buffer comprise 125 mM sodium chloride.

In one embodiment at least one product-related impurity is a homodimer, high molecular weight species, half antibody, aggregate, low molecular weight species, antibody fragment, or a light chain mis-assembly. In one embodiment a purification process that includes a unit operation comprising cation exchange chromatography performed according to the method described above.

In one embodiment the method above further comprises, before and/or after the cation exchange chromatography step, one or more unit operations for purifying the multispecific protein, comprising affinity chromatography, ion exchange chromatography, hydrophobic interaction chromatography column, and/or mixed-mode chromatography column.

In one embodiment the multispecific protein is a bispecific protein. In one embodiment the multispecific protein is a bispecific antibody. In one embodiment a purified, multispecific protein produced according to the method described above. In one embodiment the cation exchange chromatography medium is a resin.

The invention provides a method for reducing low pI product-related impurities in the eluate from cation exchange chromatography, the method comprising loading a composition comprising a multispecific protein and at least one product-related impurity having a pI lower than the multispecific protein, onto a cation exchange chromatography medium; washing the cation exchange medium with a first wash buffer, washing the cation exchange medium with a second wash buffer comprises 100-147 mM sodium chloride; eluting the multispecific protein from the cation exchange chromatography resin; wherein the cation exchange chromatography eluate has reduced low pI product-related impurities compared to the cation exchange chromatography eluate recovered in a corresponding method in which no sodium chloride is included in a wash buffer formulation. In one embodiment the cation exchange medium is washed with a third wash buffer.

The invention provides a method for performing cation exchange chromatography under high salt wash conditions to reduce product-related impurities, the method comprising loading a composition comprising a multispecific protein and at least one product-related impurity onto an equilibrated cation exchange column; washing the cation exchange medium with at least two wash buffers, wherein one wash buffer comprising 100-147 mM sodium chloride; and eluting the bound multispecific protein from the cation exchange chromatography resin. In one embodiment prior to loading the composition, the cation exchange medium is equilibrated with a buffer that contains no sodium chloride.

The invention provides a method for producing an isolated, purified, recombinant multispecific protein, the method comprising establishing a cell culture in a bioreactor with a host cell expressing the multispecific protein; culturing the host cells to express the multispecific protein; harvesting the recombinant multispecific protein from the cell culture; affinity purifying the recombinant multispecific protein; loading the affinity purified recombinant multispecific protein onto a cation exchange chromatography resin; washing the cation exchange resin with at least one wash comprising 100-147 mM sodium chloride; eluting the multispecific protein from the cation exchange chromatography resin; and loading the cation exchange chromatography eluate comprising the recombinant multispecific protein onto an additional chromatography medium in flow through mode. In one embodiment the additional chromatography medium is selected from cation exchange chromatography medium, multi-modal chromatography medium, hydrophobic interaction chromatography medium, and hydroxyapatite chromatography medium. In one embodiment the affinity purified multispecific protein is in an eluate pool and is subjected to low pH viral inactivation, followed by neutralization, prior to loading onto the cation exchange medium. In one embodiment the flow through from the third chromatography medium is subjected to an ultrafiltration and diafiltration unit operation. In one embodiment an isolated, purified, recombinant multispecific protein is made according to the method described above. In one embodiment a pharmaceutical composition comprising the isolated, purified, recombinant multispecific protein produced with the method described above.

### BRIEF DESCRIPTION OF THE DRAWINGS

FIG. 1 Shows product-related impurities (homodimer, NCG) eluting with the main product, Bi-specific #1.

FIG. 2 Shows that following high salt wash conditions, all the product-related impurities that were lower in pI than the main product were washed off the column between the second and third wash steps. The baseline returned to zero before the elution. The elution peak was reduced to one peak.

FIG. 3 Shows that multiple impurities remained on the column and were eluted with the main product. These impurities included half antibodies (fractions 1-4 with ˜50% half antibodies), 2× light chain-mis-assemblies (Fractions 5 and 6), and high molecular weight (fractions 12-21).

FIG. 4 Shows that the high salt wash resulted in a reduced number of peaks in the elution profile, from four peaks to a single peak with a small shoulder that still contained 2× LC2 mispaired species, (LC1/LC2<0.11 compared to expected ratio of 1).

FIG. 5 Shows one elution peak resulting from the high load density at a steep elution gradient. The low pI product-related impurities did not resolve from the main product under the high load density and are mostly in fractions 1-3 as shown by a reduced CE-SDS.

FIG. 6 Shows lowering loading density with a shallower gradient resulted in separation of the main low pI product-impurities into a distinct peak containing mispaired LC1 species.

FIG. 7 Shows the results of the high salt wash. There was a reduction in the number of impurity peaks in the elution profile, the majority of the impurities were removed during the second and third wash steps. A third wash step reestablished the UV baseline to zero before the start of the elution, tightening the elution profile, resulting in a much more efficient collection and better quality of the main product.

FIG. 8 Shows that following high salt wash conditions, all the low pI product-related impurities (homodimer species with pI of 6.8 and any aggregated species with low pI) flowed through the column into the waste or were collected separately, in a “wash pool”, to test their content. The baseline returned to zero before the elution. The elution peak was reduced to one peak.

### DETAILED DESCRIPTION OF THE INVENTION

Because there is not much information in the literature relating to downstream processing of multispecific proteins, platforms developed for monoclonal antibodies are often applied (Shulka and Norman, Chapter 26 Downstream Processing of Fc Fusion Proteins, Bispecific Antibodies, and Antibody-Drug Conjugates, in Process Scale Purification of Antibodies Second Edition, Uwe Gottswchalk editor, p 559-594, John Wiley & Sons, 2017). Multispecific proteins are highly engineered and subjecting such proteins to a cation exchange chromatography (CEX) in bind and elute mode under conditions typical for antibodies may not be sufficient to stabilize the multispecific proteins and/or manage the product-related impurities that elute with the main product. These product-related impurities have isoelectric points that are both lower and higher than the main product. Those product-related impurities with pIs lower than the main product were found to elute with the main product, as pre-peaks. Such an elution profile does not support the development of a robust, sustainable, commercial scale manufacturing process.

It was found that use of a high salt wash strategy resulted in better yield and purity of the main product and improved the manufacturing process. Addition of a high salt wash reduced the lower pI product-related impurities in the eluate pool by removing them prior to the elution step. The addition of a wash step with sodium chloride in a range of 105-147 mM resulted in product-related impurities with pIs that were lower than the main product being washed off, or eluted from, the cation exchange medium before the elution step, reducing the number of peaks in the elution profile. Automatic pooling of eluate based on OD becomes difficult when pre-peaks are present. It was found that incorporating a high salt wash step into the cation exchange chromatography protocol reduced the pre-peaks associated with the low pI product-related impurities prior to elution, lessening the need for a more conservative strategy to be used during collection of the main product during elution, which would likely result in lower yields and a less robust manufacturing process.

The invention provides a method for purifying a multispecific protein comprising loading a sample comprising a multispecific protein onto a cation exchange chromatography medium; washing the cation exchange medium with at least one wash buffer comprising 100-147 mM sodium chloride; and eluting the multispecific protein from the cation exchange chromatography resin.

The invention provides a method for reducing low pI product-related impurities in the eluate from cation exchange chromatography, the method comprising loading a composition comprising a multispecific protein and at least one product-related impurity having a pI lower than the multispecific protein, onto a cation exchange chromatography medium; washing the cation exchange medium with a first wash buffer, washing the cation exchange medium with a second wash buffer comprises 100-147 mM sodium chloride; eluting the multispecific protein from the cation exchange chromatography resin; wherein the cation exchange chromatography eluate has reduced low pI product-related impurities compared to the cation exchange chromatography eluate recovered in a corresponding method in which no sodium chloride is included in a wash buffer formulation.

The invention provides a method for performing cation exchange chromatography under high salt wash conditions to reduce product-related impurities, the method comprising loading a composition comprising a multispecific protein and at least one product-related impurity onto an equilibrated cation exchange column; washing the cation exchange medium with at least two wash buffers, wherein one wash buffer comprising 100-147 mM sodium chloride; and eluting the bound multispecific protein from the cation exchange chromatography resin.

The invention provides a method for producing an isolated, purified, recombinant multispecific protein, the method comprising establishing a cell culture in a bioreactor with a host cell expressing the multispecific protein; culturing the host cells to express the multispecific protein; harvesting the recombinant multispecific protein from the cell culture; affinity purifying the recombinant multispecific protein; loading the affinity purified recombinant multispecific protein onto a cation exchange chromatography resin; washing the cation exchange resin with at least one wash comprising 100-147 mM sodium chloride; eluting the multispecific protein from the cation exchange chromatography resin; and loading the cation exchange chromatography eluate comprising the recombinant multispecific protein onto a third chromatography medium in flow through mode.

The invention provides a purification process that includes a unit operation comprising cation exchange chromatography preformed according to the methods disclosed herein.

The invention provides a purified, multispecific protein produced according to the methods disclosed herein.

The invention provides a pharmaceutical composition comprising the isolated, purified, recombinant multispecific protein produced according to any of the methods described herein.

In one embodiment at least one wash buffer comprises 0-147 mM sodium chloride. In one embodiment at least one wash buffer comprises 70-147 mM sodium chloride. In one embodiment at least one wash buffer comprises 100-147 mM sodium chloride. In one embodiment at least one wash buffer comprises 100-125 mM sodium chloride. In one embodiment at least one wash buffer comprises 100-105 mM sodium chloride. In one embodiment at least one wash buffer comprises 105-147 mM sodium chloride. In one embodiment at least one wash buffer comprises 105-125 mM sodium chloride. In one embodiment at least one wash buffer comprises 125-147 mM sodium chloride. In one embodiment at least one wash buffer comprises 0, 70, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 142, 144, 145, 146, or 147 mM sodium chloride. In one embodiment at least one wash buffer comprises 0 mM sodium chloride. In one embodiment at least one wash buffer comprises 70 mM sodium chloride. In one embodiment at least one wash buffer comprises 100 mM sodium chloride. In one embodiment at least one wash buffer comprises 105 mM sodium chloride. In one embodiment at least one wash buffer comprises 125 mM sodium chloride. In one embodiment at least one wash buffer comprises 147 mM sodium chloride.

In one embodiment of the invention, at least one wash buffer comprises acetate. In one embodiment at least one wash buffer comprises acetate, pH of 5.0±0.05 to 5.0±0.1. In one embodiment at least one wash buffer comprises acetate, pH 4.9-5.1. In one embodiment at least one wash buffer comprises acetate at pH, 4.9, 5.0, or 5.1. In one embodiment at least one wash buffer comprises 100 mM acetate. In one embodiment at least one wash buffer comprises acetate, pH 5.0±0.05% to pH 5.0±0.1%. In one embodiment at least one wash buffer comprises acetate, pH 4.9-5.1. In one embodiment at least one wash buffer comprises acetate at pH, 4.9, 5.0, or 5.1. In one embodiment at least one wash buffer comprises acetate at pH, 5.0. Wash buffers can be 0.05 to 0.1 pH unit higher and lower with variations in conductivity to robustly remove product-related impurities.

In one embodiment at least one wash buffer comprises acetate, 0-147 mM sodium chloride. In one embodiment at least one wash buffer comprises acetate, 70-147 mM sodium chloride. In one embodiment at least one wash buffer comprises acetate, 100-147 mM sodium chloride. In one embodiment at least one wash buffer comprises acetate, 100-125 mM sodium chloride. In one embodiment at least one wash buffer comprises acetate, 100-105 mM sodium chloride. In one embodiment at least one wash buffer comprises acetate, 105-147 mM sodium chloride. In one embodiment at least one wash buffer comprises acetate, 105-125 mM sodium chloride. In one embodiment at least one wash buffer comprises acetate, 125-147 mM sodium chloride. In a related embodiment, the acetate concentration is 100 mM.

In one embodiment at least one wash buffer comprises acetate, 0 mM sodium chloride. In one embodiment at least one wash buffer comprises acetate, 70 mM sodium chloride. In one embodiment at least one wash buffer comprises acetate, 100 mM sodium chloride. In one embodiment at least one wash buffer comprises acetate, 105 mM sodium chloride. In one embodiment at least one wash buffer comprises acetate, 125 mM sodium chloride. In one embodiment at least one wash buffer comprises acetate, 147 mM sodium chloride. In a related embodiment, the acetate concentration is 100 mM.

In one embodiment at least one wash buffer comprises acetate, 0-147 mM sodium chloride, pH 5.0±0.05% to pH 5.0±0.1%. In one embodiment at least one wash buffer comprises acetate, 70-147 mM sodium chloride, pH 5.0±0.05% to pH 5.0±0.1%. In one embodiment at least one wash buffer comprises acetate, 100-147 mM sodium chloride, pH 5.0±0.05% to pH 5.0±0.1%. In one embodiment at least one wash buffer comprises acetate, 100-125 mM sodium chloride, pH 5.0±0.05% to pH 5.0±0.1%. In a related embodiment at least one wash buffer comprises acetate, 100-105 mM sodium chloride, pH 5.0±0.05% to pH 5.0±0.1%. In a related embodiment at least one wash buffer comprises acetate, 105-147 mM sodium chloride, pH 5.0±0.05% to pH 5.0±0.1%. In a related embodiment at least one wash buffer comprises acetate, 105-125 mM sodium chloride, pH 5.00±0.05% to pH 5.0±0.1%. In a related embodiment at least one wash buffer comprises acetate, 125-147 mM sodium chloride, pH 5.00±0.05% to pH 5.0±0.1%. In a related embodiment, the acetate concentration is 100 mM.

In a related embodiment at least one wash buffer comprises acetate, 0 mM sodium chloride, pH 5.00±0.05% to pH 5.0±0.1%. In a related embodiment at least one wash buffer comprises acetate, 70 mM sodium chloride, pH 5.00±0.05% to pH 5.0±0.1%. In a related embodiment at least one wash buffer comprises acetate, 100 mM sodium chloride, pH 5.00±0.05% to pH 5.0±0.1%. In a related embodiment at least one wash buffer comprises acetate, 105 mM sodium chloride, pH 5.00±0.05% to pH 5.0±0.1%. In a related embodiment at least one wash buffer comprises acetate, 125 mM sodium chloride, pH 5.00±0.05% to pH 5.0±0.1%. In a related embodiment at least one wash buffer comprises acetate, 147 mM sodium chloride, pH 5.00±0.05% to pH 5.0±0.1%. In a related embodiment, the acetate concentration is 100 mM.

In a related embodiment at least one wash buffer comprises acetate, 0-147 mM sodium chloride, pH 4.9-5.1. In a related embodiment at least one wash buffer comprises acetate, 70-147 mM sodium chloride, pH 4.9-5.1. In a related embodiment at least one wash buffer comprises acetate, 100-147 mM sodium chloride, pH 4.9-5.1. In one embodiment at least one wash buffer comprises acetate, 100-125 mM sodium chloride, pH 4.9-5.1. In a related embodiment at least one wash buffer comprises acetate, 100-105 mM sodium chloride, pH 4.9-5.1. In a related embodiment at least one wash buffer comprises acetate, 105-147 mM sodium chloride, pH 4.9-5.1. In a related embodiment at least one wash buffer comprises acetate, 105-125 mM sodium chloride, pH 4.9-5.1. In a related embodiment at least one wash buffer comprises acetate, 125-147 mM sodium chloride, pH 4.9-5.1. In a related embodiment, the acetate concentration is 100 mM.

In a related embodiment at least one wash buffer comprises acetate, 0 mM sodium chloride, pH 4.9-5.1. In a related embodiment at least one wash buffer comprises acetate, 70 mM sodium chloride, pH 4.9-5.1. In a related embodiment at least one wash buffer comprises acetate, 100 mM sodium chloride, pH 4.9-5.1. In a related embodiment at least one wash buffer comprises acetate, 105 mM sodium chloride, pH 4.9-5.1. In a related embodiment at least one wash buffer comprises acetate, 125 mM sodium chloride, pH 4.9-5.1. In a related embodiment at least one wash buffer comprises acetate, 147 mM sodium chloride, pH 4.9-5.1. In a related embodiment, the acetate concentration is 100 mM.

In a related embodiment at least one wash buffer comprises acetate, 0-147 mM sodium chloride, pH 4.9, 5.0 or 5.1. In a related embodiment at least one wash buffer comprises acetate, 70-147 mM sodium chloride, pH 4.9, 5.0 or 5.1. In a related embodiment at least one wash buffer comprises acetate, 100-147 mM sodium chloride, pH 4.9, 5.0 or 5.1. In one embodiment at least one wash buffer comprises acetate, 100-125 mM sodium chloride, pH 4.9, 5.0 or 5.1. In a related embodiment at least one wash buffer comprises acetate, 100-105 mM sodium chloride, pH 4.9, 5.0 or 5.1. In a related embodiment at least one wash buffer comprises acetate, 105-147 mM sodium chloride, pH 4.9, 5.0 or 5.1. In a related embodiment at least one wash buffer comprises acetate, 105-125 mM sodium chloride, pH 4.9, 5.0 or 5.1. In a related embodiment at least one wash buffer comprises acetate, 125-147 mM sodium chloride, pH 4.9, 5.0 or 5.1. In a related embodiment, the acetate concentration is 100 mM.

In a related embodiment at least one wash buffer comprises acetate, 0 mM sodium chloride, pH 4.9, 5.0 or 5.1. In a related embodiment at least one wash buffer comprises acetate, 70 mM sodium chloride, pH 4.9, 5.0 or 5.1. In a related embodiment at least one wash buffer comprises acetate, 100 mM sodium chloride, pH 4.9, 5.0 or 5.1. In a related embodiment at least one wash buffer comprises acetate, 105 mM sodium chloride, pH 4.9, 5.0 or 5.1. In a related embodiment at least one wash buffer comprises acetate, 125 mM sodium chloride, pH 4.9, 5.0 or 5.1. In a related embodiment at least one wash buffer comprises acetate, 147 mM sodium chloride, pH 4.9, 5.0 or 5.1. In a related embodiment, the acetate concentration is 100 mM.

In one embodiment, at least one wash buffer comprises 100 mM acetate, 100 mM sodium chloride, pH 5.0±0.5% to pH 5.0±0.1. In one embodiment, at least one wash buffer comprises 100 mM acetate, 105 mM sodium chloride, pH 5.0±0.5% to pH 5.0±0.1. In one embodiment, at least one wash buffer comprises 100 mM acetate, 125 mM sodium chloride, pH 5.0±0.5% to 0.1. In one embodiment, at least one wash buffer comprises 100 mM acetate, 147 mM sodium chloride, pH 5.0±0.5% to pH 5.0±0.1. In one embodiment of the invention, at least one wash buffer comprises 100 mM acetate, 70 mM sodium chloride, pH 5.0±0.5% to pH 5.0±0.1. In one embodiment of the invention, at least one wash buffer comprises 100 mM acetate, 0 mM sodium chloride, pH 5.0±0.5% to pH 5.0±0.1.

In one embodiment of the invention, at least one wash buffer comprises 100 mM acetate, 0-147 mM sodium chloride, pH 5.0. In one embodiment of the invention, at least one wash buffer comprises 100 mM acetate, 70-147 mM sodium chloride, pH 5.0. In one embodiment of the invention, at least one wash buffer comprises 100 mM acetate, 100-125 mM sodium chloride, pH 5.0. In one embodiment of the invention, at least one wash buffer comprises 100 mM acetate, 100-105 mM sodium chloride, pH 5.0. In one embodiment of the invention, at least one wash buffer comprises 100 mM acetate, 105-147 mM sodium chloride, pH 5.0. In one embodiment of the invention, at least one wash buffer comprises 100 mM acetate, 105-125 mM sodium chloride, pH 5.0. In one embodiment of the invention, at least one wash buffer comprises 100 mM acetate, 125-147 mM sodium chloride, pH 5.0. Wash buffers can be 0.05 to 0.1 pH unit higher and lower with variations in conductivity to robustly remove product-related impurities.

In one embodiment the cation exchange medium is washed with at least two wash buffers. In one embodiment, the wash buffers comprise 0-147 mM sodium chloride. In one embodiment at least one wash buffer comprises 0, 70, 100, 105, 125, or 147 mM sodium chloride. In one embodiment the wash buffers comprise acetate. In one embodiment the wash buffer comprises 100 mM acetate. In one embodiment the pH of at least one wash buffer is 5.0±0.05% to pH 5.0±0.1%. In one embodiment the pH at least one wash buffer is 4.9-5.1. In one embodiment the pH at least one wash buffer is 4.9, 5.0, or 5.1. In one embodiment the cation exchange medium is washed with at least two wash buffers, at least one wash buffer comprises acetate, 0-70 mM sodium chloride, and at least one wash buffer comprises acetate, 100-147 mM sodium. In one embodiment the cation exchange chromatography medium is washed with at least two wash buffers, one wash buffer comprises acetate, 0 mM sodium chloride, followed by a wash buffer comprising acetate, 100-147 mM sodium chloride. In one embodiment the cation exchange chromatography medium is washed with at least two wash buffers, one wash buffer comprises acetate, 100-147 mM sodium chloride, followed by a wash buffer comprising acetate, 0-70 mM sodium chloride. In one embodiment the cation exchange chromatography medium is washed with at least two wash buffers, the first wash buffer comprises acetate, 0 mM NaCl, and the second wash buffer comprises acetate, 100-147 mM sodium chloride. In one embodiment, the sodium chloride concentration of the second wash buffer is selected from 100, 105, 125, and 147 mM sodium chloride. In one embodiment the cation exchange chromatography medium is washed with at least two wash buffers, the first wash buffer comprises acetate, 100-147 mM NaCl, and the second wash buffer comprises acetate, 0-70 mM sodium chloride. In one embodiment, the sodium chloride concentration of the first wash buffer is selected from 100, 105, and 147 mM sodium chloride. In one embodiment the concentration of the first buffer concentration is 100 mM acetate, 0 mM sodium chloride, and the concentration of the second wash buffer is 100 mM acetate, 125 mM sodium chloride

In one embodiment the cation exchange medium is washed with at least three wash buffers. In one embodiment, the wash buffers comprise 0-147 mM sodium chloride. In one embodiment at least one wash buffer comprises 0, 70, 100, 105, 125, or 147 mM sodium chloride. In one embodiment the wash buffer comprises acetate. In one embodiment at least one wash buffer comprises 100 mM acetate. In one embodiment the pH of at least one wash buffer is 5.0±0.05% to pH 5.0±0.1%. In one embodiment the pH of at least one wash buffer is 4.9-5.1. In one embodiment the pH of at least wash buffer is 4.9, 5.0, or 5.1.

In one embodiment the cation exchange medium is washed with at least three wash buffers, at least two of the wash buffers comprise acetate, 0-70 mM sodium chloride, and at least one wash buffer comprises acetate, 100-147 mM sodium. In one embodiment the cation exchange chromatography medium is washed with at least three wash buffers, one wash buffer comprises acetate, 0 mM sodium chloride; followed by a wash buffer comprising acetate, 100-147 mM sodium chloride; followed by a wash buffer comprising acetate, 0-70 mM sodium chloride. In one embodiment, a first wash buffer comprises acetate, 0 mM NaCl; a second wash buffer comprises acetate, 100-147 mM sodium chloride; and a third wash buffer comprises acetate, 0-70 mM sodium chloride. In one embodiment, the sodium chloride concentration of the first wash buffer is 0 mM sodium chloride. In one embodiment, the sodium chloride concentration of the second wash buffer is selected from 100, 105, and 147 mM sodium chloride. In one embodiment, the sodium chloride concentration of the third wash buffer is selected from 0 and 70 mM sodium chloride. In one embodiment the first wash buffer concentration is 100 mM acetate, 0 mM sodium chloride; the second wash buffer is selected from 100 mM acetate, 100 mM sodium chloride and 100 mM acetate, 105 mM sodium chloride; and the third wash buffer is 100 mM acetate, 0 mM sodium chloride. In one embodiment the first wash buffer is 100 mM acetate, 0 mM sodium chloride; the second wash buffer is 100 mM acetate, 147 mM sodium chloride and the third wash buffer is 100 mM acetate, 70 mM sodium chloride.

In one embodiment the cation exchange resin is washed with 2.5 mM/CV of a wash buffer comprising 147 mM sodium chloride.

In one embodiment the multispecific protein is eluted from the cation exchange medium by a gradient. In a related embodiment the gradient is a linear or step gradient. In a related embodiment the gradient is a salt gradient. In a related embodiment at least one of the buffers used to form the elution gradient comprises 0-1M sodium chloride. In a related embodiment at least one of the buffers used to form the elution gradient comprises 70-500 mM sodium chloride.

In one embodiment at least one of the buffers used to form the elution gradient comprises 125 mM sodium chloride. In a related embodiment at least one wash buffer and one elution buffer comprise 125 mM sodium chloride.

In one embodiment prior to loading the composition, the cation exchange medium is equilibrated with a buffer that contains no sodium chloride.

In one embodiment the cation exchange medium is loaded with 10 g/L of the multispecific protein, washed with a wash buffer comprising 105 mM sodium chloride and eluted in a salt gradient at 8 mM/CV.

In one embodiment at least one product-related impurity is a homodimer, high molecular weight species, half antibody, aggregate, low molecular weight species, antibody fragment, or a light chain mis-assembly.

In one embodiment the affinity purified multispecific protein is in an eluate pool and is subjected to low pH viral inactivation, followed by neutralization, prior to loading onto the cation exchange medium.

In one embodiment the cation exchange chromatography medium is a resin.

In one embodiment the third chromatography medium is selected from cation exchange chromatography medium, multi-modal chromatography medium, hydrophobic interaction chromatography medium, and hydroxyapatite chromatography medium. In one embodiment the flow through from the third chromatography medium is subjected to an ultrafiltration and diafiltration unit operation.

In one embodiment is provided before and/or after the cation exchange chromatography step, one or more unit operations for purifying the multispecific protein, comprising affinity chromatography, ion exchange chromatography, hydrophobic interaction chromatography column, and/or mixed-mode chromatography column.

In one embodiment the multispecific protein is a bispecific protein. In one embodiment the multispecific protein is a bispecific antibody.

“Multispecific”, “multispecific protein”, and “multispecific antibody” are used herein to refer to proteins that are recombinantly engineered to simultaneously bind and neutralize at least two different antigens or at least two different epitopes on the same antigen. For example, multispecific proteins may be engineered to target immune effectors in combination with targeting cytotoxic agents to tumors or infectious agents. These multispecific proteins have been found useful for a variety of applications, such as in cancer immunotherapy, by redirecting immune effector cells to tumor cells, modifying cell signaling by blocking signaling pathways, targeting tumor angiogenesis, blocking cytokines, and as pre-targeted delivery vehicles for drugs, such as delivery of chemotherapeutic agents, radiolabels (to improve detection sensitivity) and nanoparticles (directed to specific cells/tissues, such as cancer cells).

The most common and most diverse group of multispecific proteins are those that bind two antigens, referred to herein as “bispecific”, “bispecific proteins”, and “bispecific antibodies”. Bispecific proteins can be grouped in two broad categories: immunoglobulin G (IgG)-like molecules and non-IgG-like molecules. IgG-like molecules retain Fc-mediated effector functions, such as antibody-dependent cell mediated cytotoxicity (ADCC), complement-dependent cytotoxicity (CDC), and antibody-dependent cellular phagocytosis (ADCP), the Fc region helps improve solubility and stability and facilitate some purification operations. Non-IgG-like molecules are smaller, enhancing tissue penetration (see Sedykh et al., Drug Design, Development and Therapy 18(12), 195-208, 2018; Fan et al., J Hematol & Oncology 8:130-143, 2015; Spiess et al., Mol Immunol 67, 95-106, 2015; Williams et al., Chapter 41 Process Design for Bispecific Antibodies in Biopharmaceutical Processing Development, Design and Implementation of Manufacturing Processes, Jagschies et al., eds., 2018, pages 837-855. Bispecific proteins are sometimes used as a framework for additional components having binding specificities to different antigens or numbers of epitopes, increasing the binding specificity of the molecule.

The formats for bispecific proteins, which include bispecific antibodies, are constantly evolving and include, but are not limited to, quadromas, knobs-in-holes, cross-Mabs, dual variable domains IgG (DVD-IgG), IgG-single chain Fv (scFv), scFv-CH3 KIH, dual action Fab (DAF), half-molecule exchange, κλ-bodies, tandem scFv, scFv-Fc, diabodies, single chain diabodies (scDiabodies), scDiabodies-CH3, triple body, miniantibody, minibody, TriBi minibody, tandem diabodies, scDiabody-HAS, Tandem scFv-toxin, dual-affinity retargeting molecules (DARTs), nanobody, nanobody-HSA, dock and lock (DNL), strand exchange engineered domain SEEDbody, Triomab, leucine zipper (LUZ-Y), XmAb®; Fab-arm exchange, DutaMab, DT-IgG, charged pair, Fcab, orthogonal Fab, IgG(H)-scFv, scFV-(H)IgG, IgG(L)-scFV, IgG(L1H1)-Fv, IgG(H)-V, V(H)-IgG, IgG(L)-V V(L)-IgG, KIH IgG-scFab, 2scFV-IgG, IgG-2scFv, scFv4-Ig, Zybody, DVI-Ig4 (four-in-one), Fab-scFv, scFv-CH-CL-scFV, F(ab′)2-scFv2, scFv-KIH, Fab-scFv-Fc, tetravalent HCAb, scDiabody-Fc, diabody-Fc, intrabody, ImmTAC, HSABody, IgG-IgG, Cov-X-Body, scFv1-PEG-scFv2, bi-specific T cell engagers (BITE®) and half-life extended bispecific T cell engagers (HLE BITE®) (Fan supra; Spiess supra; Sedykh supra; Seimetz et al., Cancer Treat Rev 36(6) 458-67, 2010; Shulka and Norman, Chapter 26 Downstream Processing of Fc Fusion Proteins, Bispecific Antibodies, and Antibody-Drug Conjugates, in Process Scale Purification of Antibodies Second Edition, Uwe Gottswchalk editor, p 559-594, John Wiley & Sons, 2017; Moore et al., MAbs 3:6, 546-557, 2011).

In some embodiments, bispecific proteins may include blinatumomab, catumaxomab, ertumaxomab, solitomab, targomiRs, lutikizumab (ABT981), vanucizumab (RG7221), remtolumab (ABT122), ozoralixumab (ATN103), floteuzmab (MGD006), pasotuxizumab (AMG112, MT112), lymphomun (FBTA05), (ATN-103), AMG211 (MT111, Medi-1565), AMG330, AMG420 (B1836909), AMG-110 (MT110), MDX-447, TF2, rM28, HER2Bi-aATC, GD2Bi-aATC, MGD006, MGD007, MGD009, MGD010, MGD011 (JNJ64052781), IMCgp100, indium-labeled IMP-205, xm734, LY3164530, OMP-305BB3, REGN1979, COV322, ABT112, ABT165, RG-6013 (ACE910), RG7597 (MEDH7945A), RG7802, RG7813(RO6895882), RG7386, BITS7201A (RG7990), RG7716, BFKF8488A (RG7992), MCLA-128, MM-111, MM141, MOR209/ES414, MSB0010841, ALX-0061, ALX0761, ALX0141; BII034020, AFM13, AFM11, SAR156597, FBTA05, PF06671008, GSK2434735, MEDI3902, MEDI0700, MEDI7352, as well as the molecules or variants or analogs thereof and biosimilars of any of the foregoing.

Multispecific proteins also include trispecific antibodies, tetravalent bispecific antibodies, multispecific proteins without antibody components such as dia-, tria- or tetrabodies, minibodies, and single chain proteins capable of binding multiple targets. Coloma, M. J., et. al., Nature Biotech. 15 (1997) 159-163

In some embodiments, multispecific proteins of interest bind, neutralize and/or interact specifically to one or more CD proteins, HER receptor family proteins, cell adhesion molecules, growth factors, nerve growth factors, fibroblast growth factors, transforming growth factors (TGF), insulin-like growth factors, osteoinductive factors, insulin and insulin-related proteins, coagulation and coagulation-related proteins, colony stimulating factors (CSFs), other blood and serum proteins blood group antigens; receptors, receptor-associated proteins, growth hormones, growth hormone receptors, T-cell receptors; neurotrophic factors, neurotrophins, relaxins, interferons, interleukins, viral antigens, lipoproteins, integrins, rheumatoid factors, immunotoxins, surface membrane proteins, transport proteins, homing receptors, addressins, regulatory proteins, and immunoadhesins.

In some embodiments multispecific proteins of interest bind, neutralize and/or interact with one or more of the following, alone or in any combination: CD proteins including but not limited to CD3, CD4, CD5, CD7, CD8, CD19, CD20, CD22, CD25, CD30, CD33, CD34, CD38, CD40, CD70, CD123, CD133, CD138, CD171, and CD174, HER receptor family proteins, including, for instance, HER2, HER3, HER4, and the EGF receptor, EGFRvIII, cell adhesion molecules, for example, LFA-1, Mol, p 150, 95, VLA-4, ICAM-1, VCAM, and alpha v/beta 3 integrin, growth factors, including but not limited to, for example, vascular endothelial growth factor (“VEGF”); VEGFR2, growth hormone, thyroid stimulating hormone, follicle stimulating hormone, luteinizing hormone, growth hormone releasing factor, parathyroid hormone, mullerian-inhibiting substance, human macrophage inflammatory protein (MIP-1-alpha), erythropoietin (EPO), nerve growth factor, such as NGF-beta, platelet-derived growth factor (PDGF), fibroblast growth factors, including, for instance, aFGF and bFGF, epidermal growth factor (EGF), Cripto, transforming growth factors (TGF), including, among others, TGF-α and TGF-β, including TGF-β1, TGF-β2, TGF-β3, TGF-β4, or TGF-β5, insulin-like growth factors-I and -II (IGF-I and IGF-II), des(1-3)-IGF-I (brain IGF-I), and osteoinductive factors, insulins and insulin-related proteins, including but not limited to insulin, insulin A-chain, insulin B-chain, proinsulin, and insulin-like growth factor binding proteins; (coagulation and coagulation-related proteins, such as, among others, factor VIII, tissue factor, von Willebrand factor, protein C, alpha-1-antitrypsin, plasminogen activators, such as urokinase and tissue plasminogen activator (“t-PA”), bombazine, thrombin, thrombopoietin, and thrombopoietin receptor, colony stimulating factors (CSFs), including the following, among others, M-CSF, GM-CSF, and G-CSF, other blood and serum proteins, including but not limited to albumin, IgE, and blood group antigens, receptors and receptor-associated proteins, including, for example, flk2/flt3 receptor, obesity (OB) receptor, growth hormone receptors, and T-cell receptors; neurotrophic factors, including but not limited to, bone-derived neurotrophic factor (BDNF) and neurotrophin-3, -4, -5, or -6 (NT-3, NT-4, NT-5, or NT-6); relaxin A-chain, relaxin B-chain, and prorelaxin, interferons, including for example, interferon-alpha, -beta, and -gamma, interleukins (ILs), e.g., IL-1 to IL-10, IL-12, IL-15, IL-17, IL-23, IL-12/IL-23, IL-2Ra, IL1-R1, IL-6 receptor, IL-4 receptor and/or IL-13 to the receptor, IL-13RA2, or IL-17 receptor, IL-1RAP; viral antigens, including but not limited to, an AIDS envelope viral antigen, lipoproteins, calcitonin, glucagon, atrial natriuretic factor, lung surfactant, tumor necrosis factor-alpha and -beta, enkephalinase, BCMA, STEAP1, IgKappa, ROR-1, ERBB2, mesothelin, RANTES (regulated on activation normally T-cell expressed and secreted), mouse gonadotropin-associated peptide, Dnase, FR-alpha, inhibin, and activin, integrin, protein A or D, rheumatoid factors, immunotoxins, bone morphogenetic protein (BMP), superoxide dismutase, surface membrane proteins, decay accelerating factor (DAF), AIDS envelope, transport proteins, homing receptors, MIC (MIC-a, MIC-B), ULBP 1-6, EPCAM, addressins, regulatory proteins, immunoadhesins, antigen-binding proteins, somatropin, CTGF, CTLA4, eotaxin-1, MUC1, CEA, c-MET, Claudin-18, GPC-3, EPHA2, FPA, LMP1, MG7, NY-ESO-1, PSCA, ganglioside GD2, ganglioside GM2, BAFF, OPGL (RANKL), myostatin, Dickkopf-1 (DKK-1), Ang2, NGF, IGF-1 receptor, hepatocyte growth factor (HGF), TRAIL-R2, c-Kit, B7RP-1, PSMA, NKG2D-1, programmed cell death protein 1 and ligand, PD1 and PDL1, mannose receptor/hCGβ, TNF, TL1A, hepatitis-C virus, mesothelin dsFv[PE38 conjugate, Legionella pneumophila (lly), IFN gamma, interferon gamma induced protein 10 (IP10), IFNAR, TALL-1, thymic stromal lymphopoietin (TSLP), proprotein convertase subtilisin/Kexin Type 9 (PCSK9), stem cell factors, Flt-3, calcitonin gene-related peptide (CGRP), OX40L, α4β7, platelet specific (platelet glycoprotein Iib/IIIb (PAC-1), transforming growth factor beta (TFGβ), Zona pellucida sperm-binding protein 3 (ZP-3), TWEAK, platelet derived growth factor receptor alpha (PDGFRα), sclerostin, and biologically active fragments or variants of any of the foregoing.

In some embodiments, multispecific proteins of interest may include bispecific antibodies that specifically bind to combinations including CD3 and CD19, EpCAM, CEA, PSA, CD33, BCMA, Her2, CD20, P-cadherin, CD123, gpA33, or B7H3. In some embodiments, bispecific antibodies of interest may include bispecific antibodies that specifically bind to combinations including IL1α+IL1β.

The multispecific proteins can be of scientific or commercial interest, particularly bispecific-based therapeutics. Multispecific proteins can be produced in various ways, most commonly by recombinant animal cell lines using cell culture methods. The multispecific proteins may be produced intracellularly or secreted into the culture medium from which it can be recovered and/or collected and may be referred to “recombinant multispecific protein”, “recombinant multispecific antibody”, “recombinant bispecific protein”, and “recombinant bispecific antibody”. The terms “isolated multispecific protein”, “isolated recombinant multispecific antibody”, “isolated bispecific protein”, and “isolated bispecific antibody”, refer to a multispecific protein, including bispecific proteins, that that have been purified away from proteins, polypeptides, DNA, and/or other contaminants or impurities such as product-related impurities, particularly low pI product-related impurities, that would interfere with its therapeutic, diagnostic, prophylactic, research, or other use. Multispecific proteins of interest include multispecific antibodies that exert a therapeutic effect by binding two or more targets, particularly targets among those listed below, including targets derived therefrom, targets related thereto, and modifications thereof.

By “purifying” is meant increasing the degree of purity of the multispecific protein in the composition by removing (partially or completely) at least one product-related impurity from the composition. Recovery and purification of multispecific proteins is accomplished by the downstream unit operations, in particular, those operations involving ion exchange chromatography, resulting in a more “homogeneous” multispecific protein composition that meets yield and product quality targets (such as reduced product-related impurities and increased product quality).

“Product-related impurity” refers to product-related variants of the multispecific protein of interest. In some instances, these impurities have a pI that is lower than the main product in an elution peak. Product-related impurities include, for example, homodimers, high molecular weight (HMW) species, half antibodies, aggregates, low molecular weight (LMW) species, antibody fragments and various combinations of antibody fragments, and light chain mis-assemblies, such as 2×LC, 3×LC, or 4×LC. “Half antibodies” refer to a product-related impurity that can form, for example, due to incomplete assembly or disruption of the interaction between the two heavy chain polypeptides. Half antibodies comprise a single light chain polypeptide and a single heavy chain polypeptide. “Homodimers” refer to a product-related impurity, that can, for example, form when heavy and light chains having specificity for the same target recombine with each other instead of pairing to form a desired bispecific heterodimer. This typically occurs during expression in the host cell. For multispecific constructs that require multiple chains (such as light chains, LCs) to pair correctly via engineered residues (such as charged paired mutations, knob-hole, etc), it is possible to still have impurities where there is a mismatch between LC and HC, wherein LC1 instead of pairing with HC1, incorrectly pairs with HC2 (2×LC1), and vice versa (2×LC2). If the multi specific protein is bivalent, having two sites for binding to each antigen of interest, it is possible to have 3×LC1, 4×LC1, and other combinations of mispaired species.

As disclosed herein, the pI of product-related impurities may be lower than the pI of the desired multispecific protein and elute with the main product. Product-related impurities having a lower pI elute prior to the main product, as pre-peaks. The “isoelectric point” or “pI” of a protein, refers to the pH at which the positive charge balances the negative charge of the protein. The pI can be calculated/determined using known methods such as from the net charge of the amino acid residues of the protein or by isoelectric focusing. In one embodiment, the difference between the pI of the product-related impurity and the main product is at least 0.5 pI unit. In another embodiment, the difference is 0.5 to 3 pI units. In another embodiment, the difference is 0.5 to 2 pI units. In another embodiment, the difference is 0.5 to 1 pI unit. In another embodiment, the difference is 1 to 3 pI units. In another embodiment, the difference is 2 to 3 pI units. In another embodiment, the difference is at least 0.5, 1, 2, 3 or more pI units.

Expression systems and constructs in the form of plasmids, expression vectors, transcription or expression cassettes that comprise one or more polynucleotides encoding a multispecific protein of interest as provided herein, as well host cells comprising such expression systems or constructs. As used herein, “vector” means any molecule or entity (e.g., nucleic acid, plasmid, bacteriophage, transposon, cosmid, chromosome, virus, virus capsid, virion, naked DNA, complexed DNA and the like) suitable for use to transfer and/or transport multispecific protein encoding information into a host cell and/or to a specific location and/or compartment within a host cell. Vectors can include viral and non-viral vectors, non-episomal mammalian vectors. Vectors are often referred to as expression vectors, for example, recombinant expression vectors and cloning vectors. The vector may be introduced into a host cell to allow replication of the vector itself and thereby amplify the copies of the polynucleotide contained therein. The cloning vectors may contain sequence components that generally include, without limitation, an origin of replication, promoter sequences, transcription initiation sequences, enhancer sequences, and selectable markers. These elements may be selected as appropriate by a person of ordinary skill in the art.

“Cell” or “Cells” include any prokaryotic or eukaryotic cell. Cells can be either ex vivo, in vitro or in vivo, either separate or as part of a higher structure such as a tissue or organ. Cells include “host cells”, also referred to as “cell lines”, which are genetically engineered to express a multispecific protein of commercial or scientific interest. Host cells are typically derived from a lineage arising from a primary culture that can be maintained in culture for an unlimited time. Genetically engineering the host cell involves transfecting, transforming or transducing the cells with a recombinant polynucleotide molecule, and/or otherwise altering (e.g., by homologous recombination and gene activation or fusion of a recombinant cell with a non-recombinant cell) to cause the host cell to express a desired recombinant multispecific protein. Methods and vectors for genetically engineering cells and/or cell lines to express a multispecific proteins of interest are well known to those of skill in the art.

A host cell can be any prokaryotic cell (for example, E. coli) or eukaryotic cell (for example, yeast, insect, or animal cells (e.g., CHO cells)). Vector DNA can be introduced into prokaryotic or eukaryotic cells via conventional transformation or transfection techniques.

Host cells, when cultured under appropriate conditions, express the multispecific protein of interest that can be subsequently collected from the culture medium (if the host cell secretes it into the medium) or directly from the host cell producing it (if it is not secreted). The selection of an appropriate host cell will depend upon various factors, such as desired expression levels, protein modifications that are desirable or necessary for activity (such as glycosylation or phosphorylation) and ease of folding into a biologically active molecule.

By “culture” or “culturing” is meant the growth and propagation of cells outside of a multicellular organism or tissue. Suitable culture conditions for mammalian cells are known in the art. Cell culture media and tissue culture media are interchangeably used to refer to media suitable for growth of a host cell during in vitro cell culture. Typically, cell culture media contains a buffer, salts, energy source, amino acids, vitamins and trace essential elements. Any media capable of supporting growth of the appropriate host cell in culture can be used. Cell culture media, which may be further supplemented with other components to maximize cell growth, cell viability, and/or recombinant protein production in a particular cultured host cell, are commercially available and include RPMI-1640 Medium, RPMI-1641 Medium, Dulbecco's Modified Eagle's Medium (DMEM), Minimum Essential Medium Eagle, F-12K Medium, Ham's F12 Medium, Iscove's Modified Dulbecco's Medium, McCoy's 5A Medium, Leibovitz's L-15 Medium, and serum-free media such as EX-CELL™ 300 Series, among others, which can be obtained from the American Type Culture Collection or SAFC Biosciences, as well as other vendors. Cell culture media can be serum-free, protein-free, growth factor-free, and/or peptone-free media. Cell culture may also be enriched by the addition of nutrients and used at greater than its usual, recommended concentrations.

Various media formulations can be used during the life of the culture, for example, to facilitate the transition from one stage (e.g., the growth stage or phase) to another (e.g., the production stage or phase) and/or to optimize conditions during cell culture (e.g. concentrated media provided during perfusion culture). A growth medium formulation can be used to promote cell growth and minimize protein expression. A production medium formulation can be used to promote production of the protein of interest and maintenance of the cells, with a minimal of new cell growth). A feed media, typically a media containing more concentrated components such as nutrients and amino acids, which are consumed during the course of the production phase of the cell culture may be used to supplement and maintain an active culture, particularly a culture operated in fed batch, semi-perfusion, or perfusion mode. Such a concentrated feed medium can contain most of the components of the cell culture medium at, for example, about 5×, 6×, 7×, 8×, 9×, 10×, 12×, 14×, 16×, 20×, 30×, 50×, 100×, 200×, 400×, 600×, 800×, or even about 1000× of their normal amount.

A growth phase may occur at a higher temperature than a production phase. For example, a growth phase may occur at a first temperature from about 35° C. to about 38° C., and a production phase may occur at a second temperature from about 29° C. to about 37° C., optionally from about 30° C. to about 36° C. or from about 30° C. to about 34° C. In addition, chemical inducers of protein production, such as, for example, caffeine, butyrate, and hexamethylene bisacetamide (HMBA), may be added at the same time as, before, and/or after a temperature shift. If inducers are added after a temperature shift, they can be added from one hour to five days after the temperature shift, optionally from one to two days after the temperature shift.

Host cells may be cultured in suspension or in an adherent form, attached to a solid substrate. Cell cultures can be established in fluidized bed bioreactors, hollow fiber bioreactors, roller bottles, shake flasks, or stirred tank bioreactors, with or without microcarriers

Cell cultures can be operated in a batch, fed batch, continuous, semi-continuous, or perfusion mode. Mammalian cells, such as CHO cells, may be cultured in bioreactors at a smaller scale of less than 100 ml to less than 1000 mls. Alternatively, larger scale bioreactors that contain 1000 mls to over 20,000 liters of media can be used. Large scale cell cultures, such as for clinical and/or commercial scale biomanufacturing of protein therapeutics, may be maintained for weeks and even months, while the cells produce the desired protein(s).

Since product-related impurities, such as homodimers, half antibodies, and the like can resemble the desired multispecific protein, strategies and techniques such as knob and hole, CrossMab, DVD IgG, and others have been developed to increase the selectivity for the desired multispecific protein during cell culture. However, there will still be a portion of product-related impurities that are produced which must be removed during downstream processing.

The resulting expressed recombinant multispecific protein can then be harvested from the cell culture media. Methods for harvesting proteins from suspension cells are known in the art and include, but are not limited to, acid precipitation, accelerated sedimentation such as flocculation, separation using gravity, centrifugation, acoustic wave separation, filtration, including membrane filtration, using ultrafilters, microfilters, tangential flow filters, alternative tangential flow, depth, and alluvial filtration filters. Recombinant proteins expressed by prokaryotes are retrieved from inclusion bodies in the cytoplasm by redox folding processes known in the art.

The harvested multispecific protein can then be purified, or partially purified, away from any impurities, such as remaining cell culture media, cell extracts, undesired components, host cell proteins, improperly expressed proteins, product-related impurities, and the like, through one or more downstream unit operations.

Purification of the multispecific protein from the harvested cell culture fluid can begin with capture chromatography. Capture chromatography makes use of mediums, such as resins, membranes, gels and the like, that will bind to the recombinant multispecific protein of interest, for example affinity chromatography, size exclusion chromatography, ion exchange chromatography, hydrophobic interaction chromatography (HIC), immobilized metal affinity chromatography (IMAC), and the like. Such materials are known in the art and are commercially available. Affinity chromatography options may comprise a substrate-binding capture mechanism, an aptamer-binding capture mechanism, or a cofactor-binding capture mechanism, for example. For multispecific proteins containing an Fc component, an antibody- or antibody fragment-binding capture mechanism such as Protein A, Protein G, Protein A/G, and Protein L can be used. The recombinant protein of interest can be tagged with a polyhistidine tag or an epitope, such a FLAG® and subsequently purified by using a specific antibody directed to such epitope.

At any point in the downstream process virus inactivation and/or virus filtration can be performed to remove viral matter from the composition comprising the multispecific protein of interest. One method for achieving virus inactivation is incubation at low pH or other suitable solution conditions for achieving the inactivation of viruses. Low pH virus inactivation can be followed with a neutralization operation that readjusts the viral inactivated solution to a pH more compatible with the requirements of the following unit operations. Typically, neutralization is at pH 5-7. Viral inactivated or neutralized viral inactivated pools may also be followed by filtration, such as depth filtration, to remove any resulting turbidity or precipitation. Viral filtration can be performed using micro- or nano-filters, such as those available from Asahi Kasei (Plavona®) and EDM Millipore (VPro®).

The term “polishing” is used herein to refer to one or more chromatographic steps performed to remove remaining contaminants and impurities such as DNA, host cell proteins, product-specific impurities, variant products and aggregates, and virus adsorption from a fluid composition comprising a recombinant multispecific protein that is close to a final desired purity. For example, polishing can be performed in bind and elute mode by passing a fluid including the recombinant multispecific protein through a chromatographic column(s) or membrane absorber(s) that selectively binds to either the target recombinant multispecific protein, or the contaminants or impurities present in the fluid composition. In such an example, the eluate/filtrate of the chromatographic column(s) or membrane absorber(s) includes the recombinant multispecific protein.

The polish chromatography makes use of a medium, such as resins and/or membranes, containing agents that can be used in either a flow-through mode (where the multispecific protein flows through the resin/membrane and is contained in the flow-through eluent while the contaminants and impurities are bound to the chromatography medium), frontal or overloaded chromatography mode (where a solution containing the protein of interest is loaded onto a column until adsorption sites on are occupied and the species with the least affinity for the stationary phase (the protein of interest) starts to elute), or bind and elute mode (where the protein of interest is bound to the chromatography medium and is eluted after the contaminants and impurities have flowed through or been washed off the chromatography medium). Examples of such chromatography operations include ion exchange chromatography (IEX), including anion exchange chromatography (AEX) and/or cation exchange chromatography (CEX); hydrophobic interaction chromatography (HIC); mixed modal or multimodal chromatography (MM), hydroxyapatite chromatography (HA); reverse phase chromatography, size exclusion chromatography (SEC), and gel filtration. In one embodiment the chromatographic method is cation exchange chromatography. In one embodiment, the cation exchange medium is a resin.

“Cation exchange chromatography” refers to chromatography performed on a solid phase medium that is negatively charged and has free cations for exchange with cations in an aqueous solution passed over or through the solid phase. The charge may be provided by attaching one or more charged ligands to the solid phase, e.g. by covalent linking. Alternatively, or in addition, the charge may be an inherent property of the solid phase (e.g. as is the case for silica, which has an overall negative charge). Commercially available cation exchange mediums are available and include but are not limited to sulphopropyl (SP) immobilized on agarose (e.g. SP-SEPHAROSE FAST FLOW™, SP-SEPHAROSE FAST FLOW XL™ or SP-SEPHAROSE HIGH PERFORMANCE™, from GE Healthcare), CAPTO S™, CAPTO SP ImpRes™, CAPTO S ImpAct™ (GE Healthcare), FRACTOGEL-SO3™, FRACTOGEL-SE HICAP™, FRACTOPREP™ (EMD Merck), Fractogel® EMD SO3-(M), Fractogel® EMD SE Hicap (M), Eshmuno® CPX, Eshmuno® S resins, Fractogel® EMD COO-(M), Mustang S Acrodisc with Mustang S AcroPrep with Mustang S, CM Ceramic HyperD® F AcroSep with CM Ceramic HyperD® F, among others.

For the inventive method, cation exchange chromatography is performed in bind and elute mode. The multispecific protein in an eluate or storage pool from a previous downstream step is loaded onto the cation exchange medium such that the multispecific protein of interest is bound to the cation exchange medium. By “binding” the multispecific protein to the cation exchange medium is meant exposing the multispecific protein to the cation exchange medium under appropriate conditions (pH/conductivity) such that the multispecific protein is reversibly immobilized in or on the cation exchange medium by virtue of ionic interactions between the multispecific protein of interest and a charged group or charged groups of the cation exchange medium. The eluate or pool may have originated from a previous unit operation, such as affinity chromatography, neutralized low pH viral inactivation, depth filtration, or a harvest and/or polish chromatography operation. Additional buffer may be added to the eluate or pool such that the final load of the multispecific protein is at a desired concentration.

The loaded cation exchange chromatography medium is then subjected to at least one wash step. Washing the cation exchange medium means passing an appropriate wash buffer through or over the cation exchange medium. A function of the wash buffer is to remove one or more contaminants from the cation exchange medium, without substantial elution of the multispecific protein of interest. By “buffer” is meant a solution that resists changes in pH by the action of its acid-base conjugate components. In one embodiment the buffer is acetate. In one embodiment is provided 100 mM acetate. In one embodiment, the pH of a wash buffer is in the range of 5.0±0.05% to 5.0±0.1%. In one embodiment, the pH of a wash buffer is in the range of 4.9 to 5.1. In one embodiment the pH of a wash buffer is 4.9, 5.0, or 5.1. In one embodiment of the invention, at least one wash buffer comprises acetate, pH 5.0. Wash buffers can be 0.05 to 0.1 pH unit higher and lower with variations in conductivity to robustly remove product-related impurities.

At least one wash buffer also comprises a salt. In one embodiment, the salt is sodium chloride. In one embodiment the concentration of sodium chloride in a wash buffer is from 0 mM to 147 mM. In one embodiment the concentration of sodium chloride in a wash buffer is from 70 mM to 147 mM. In one embodiment, the concentration of sodium chloride in a wash buffer is 100 mM to 147 mM. In one embodiment, the concentration of sodium chloride in a wash buffer is 100 mM to 125 mM. In one embodiment, the concentration of sodium chloride in a wash buffer is 100 mM to 105 mM. In one embodiment, the concentration of sodium chloride in a wash buffer is 105 mM to 147 mM. In one embodiment, the concentration of sodium chloride in a wash buffer is 105 mM to 125 mM. In one embodiment, the concentration of sodium chloride in a wash buffer is 125 mM to 147 mM. In one embodiment, the concentration of sodium chloride in a wash buffer is 0 mM. In one embodiment, the concentration of sodium chloride in a wash buffer is 70 mM. In one embodiment, the concentration of sodium chloride in a wash buffer is 100 mM. In one embodiment, the concentration of sodium chloride in a wash buffer is 105 mM. In one embodiment, the concentration of sodium chloride in a wash buffer is 125 mM. In one embodiment, the concentration of sodium chloride in a wash buffer is 147 mM.

A wash step washes the cation exchange medium following loading and prior to eluting the multispecific protein of interest. The invention provides at least one wash step comprising a high salt concentration wash buffer. The high salt wash buffer was found to wash or elute low pI product-related impurities from the cation exchange medium prior to elution of the main product. In addition to the high salt wash step, there may be one or more additional wash steps that use buffers containing no salt and/or buffers that contain salt at a lower concentration compared to a high salt wash buffer. Preferably the UV baseline has returned to or is very near to zero, at the end of the last wash step prior to the start of the elution. According to one embodiment of the invention there are at least two wash steps. In one embodiment there is a “first wash buffer” and a “second wash buffer”. According to one embodiment of the invention there are at least three wash steps. In one embodiment there is a “first wash buffer”, a “second wash buffer”, and a “third wash buffer”. The terms “first wash”, “second wash”, and/or “third wash” should not be interpreted as excluding the use of one or more additional washes or other buffers between the one or more of the wash steps. The wash buffer is used to wash or re-equilibrate the cation exchange material prior to eluting the multispecific protein of interest. One or more of the wash buffer formulations may be the same as the equilibration and/or final conditioned load buffer formulations.

In one embodiment of the invention, the first wash comprises a wash buffer comprising acetate, pH 5.0±0.5 to pH 5.0±0.1%. In one embodiment of the invention, the first wash buffer comprises acetate at a pH of 4.9 to 5.1. In one embodiment of the invention, the first wash buffer comprises acetate at a pH of 4.9, 5.0, or 5.1. In one embodiment of the invention, the first wash buffer comprises acetate at pH of 5.0. In one embodiment of the invention, the first wash buffer comprises 100 mM acetate. In one embodiment of the invention, the first wash buffer comprises 100 mM acetate, pH 5.0. In one embodiment of the invention, the first wash buffer comprises acetate, 0-147 mM sodium chloride. In one embodiment of the invention, the first wash comprises a wash buffer comprising 100 mM acetate, 0 mM sodium chloride, pH 5.0±0.5 to pH 5.0±0.1%.

In one embodiment of the invention, the second wash comprises a wash buffer comprising acetate, pH 5.0±0.5 to pH 5.0±0.1%. In one embodiment of the invention, the second wash buffer comprises acetate at a pH of 4.9 to 5.1. In one embodiment of the invention, the second wash buffer comprises acetate at a pH of 4.9, 5.0, or 5.1. In one embodiment of the invention, the second wash buffer comprises 100 mM acetate. In one embodiment of the invention, the second wash buffer comprises 100 mM acetate, pH 5.0.

In a related embodiment of the invention, the second wash buffer comprises 0-147 mM sodium chloride. In a related embodiment of the invention, the second wash buffer comprises 70-147 mM sodium chloride. In a related embodiment of the invention, the second wash buffer comprises 100-147 mM sodium chloride. In a related embodiment of the invention, the second wash buffer comprises 100-125 mM sodium chloride. In a related embodiment of the invention, the second wash buffer comprises 100-105 mM sodium chloride. In a related embodiment of the invention, the second wash buffer comprises 105-147 mM sodium chloride. In a related embodiment of the invention, the second wash buffer comprises 105-125 mM sodium chloride. In a related embodiment of the invention, the second wash buffer comprises 125-147 mM sodium chloride. In a related embodiment of the invention, the second wash buffer comprises 125 mM sodium chloride.

In one embodiment of the invention, the second wash buffer comprises 100 mM acetate, 0-147 mM sodium chloride, pH 5.0±0.05 to pH 5.0±0.1. In one embodiment of the invention, the second wash buffer comprises 100 mM acetate, 70-147 mM sodium chloride, pH 5.0±0.05 to pH 5.0±0.1. In one embodiment of the invention, the second wash buffer comprises 100 mM acetate, 100-147 mM sodium chloride, pH 5.0±0.05 to pH 5.0±0.1. In one embodiment of the invention, the second wash buffer comprises 100 mM acetate, 100-125 mM sodium chloride, pH 5.0±0.05 to pH 5.0±0.1. In one embodiment of the invention, the second wash buffer comprises 100 mM acetate, 100-105 mM sodium chloride, pH 5.0±0.05 to pH 5.0±0.1. In one embodiment of the invention, the second wash buffer comprises 100 mM acetate, 105-147 mM sodium chloride, pH 5.0±0.05 to pH 5.0±0.1. In a related embodiment, the second wash buffer comprises 100 mM acetate, 105-125 mM sodium chloride, pH 5.0±0.05 to pH 5.0±0.1. In a related embodiment, the second wash buffer comprises 100 mM acetate, 125-147 mM sodium chloride, pH 5.0±0.05 to pH 5.0±0.1. In a related embodiment, the second wash buffer comprises 100 mM acetate, 125 mM sodium chloride, pH 5.0±0.05 to pH 5.0±0.1.

In a related embodiment, the second wash buffer comprises 100 mM acetate, 0 mM sodium chloride, pH 5.0. In a related embodiment, the second wash buffer comprises 100 mM acetate, 70 mM sodium chloride, pH 5.0. In a related embodiment, the second wash buffer comprises 100 mM acetate, 100 mM sodium chloride, pH 5.0. In a related embodiment, the second wash buffer comprises 100 mM acetate, 105 mM sodium chloride, pH 5.0. In a related embodiment, the second wash buffer comprises 100 mM acetate, 125 mM sodium chloride, pH 5.0. In a related embodiment, the second wash buffer comprises 100 mM acetate, 147 mM sodium chloride, pH 5.0.

In one embodiment of the invention, the third wash comprises a wash buffer comprising acetate, pH 5.0±0.05% to pH 5.0±0.1%. In one embodiment of the invention, the third wash buffer comprises acetate at a pH of 4.9 to 5.1. In one embodiment of the invention, the third wash buffer comprises 100 mM acetate. In one embodiment of the invention, the third wash buffer comprises 100 mM acetate, pH 5.0±0.5% to pH 5.0±0.1. In one embodiment of the invention, the third wash buffer comprises 0-147 mM sodium chloride. In one embodiment of the invention, the third wash buffer comprises 0 mM sodium chloride. In one embodiment of the invention, the third wash buffer comprises 100 mM acetate, 0 mM sodium chloride, pH 5.0±0.5% to pH 5.0±0.1. In one embodiment of the invention, the third wash buffer comprises 70 mM sodium chloride. In one embodiment of the invention, the third wash buffer comprises 100 mM acetate, 70 mM sodium chloride, pH 5.0±0.5% to pH 5.0±0.1.

In one embodiment the cation exchange chromatography medium is washed with at least three wash buffers, one wash buffer comprises acetate, 0 mM sodium chloride; followed by a wash buffer comprising acetate, 100-147 mM sodium chloride; followed by a wash buffer comprising acetate, 0-70 mM sodium chloride. In one embodiment, a first wash buffer comprises acetate, 0 mM NaCl; a second wash buffer comprises acetate, 100-147 mM sodium chloride; and a third wash buffer comprises acetate, 0-70 mM sodium chloride. In one embodiment, the sodium chloride concentration of the first wash buffer is 0 mM sodium chloride. In one embodiment, the sodium chloride concentration of the second wash buffer is selected from 100, 105, and 147 mM sodium chloride. In one embodiment, the sodium chloride concentration of the third wash buffer is selected from 0 and 70 mM sodium chloride. In one embodiment the first wash buffer concentration is 100 mM acetate, 0 mM sodium chloride; the second wash buffer is selected from 100 mM acetate, 100 mM sodium chloride and 100 mM acetate, 105 mM sodium chloride; and the third wash buffer is 100 mM acetate, 0 mM sodium chloride. In one embodiment the first wash buffer is 100 mM acetate, 0 mM sodium chloride; the second wash buffer is 100 mM acetate, 147 mM sodium chloride; and the third wash buffer is 100 mM acetate, 70 mM sodium chloride.

In one embodiment of the invention, the cation exchange medium is loaded with at least 10 g/L of the multispecific protein. In one embodiment of the invention, the cation exchange medium is loaded with 10 g/L to 40 g/L of the multispecific protein. In a related embodiment of the invention, the cation exchange medium is loaded with 15 g/L to 40 g/L of the multispecific protein. In a related embodiment of the invention, the cation exchange medium is loaded with 20 g/L to 40 g/L of the multispecific protein. In a related embodiment of the invention, the cation exchange medium is loaded with 25 g/L to 40 g/L of the multispecific protein. In a related embodiment of the invention, the cation exchange medium is loaded with 35 g/L to 40 g/L of the multispecific protein. In a related embodiment, the cation exchange medium is loaded with 15 g/L to 35 g/L of the multispecific protein. In a related embodiment, the cation exchange medium is loaded with 15 g/L to 25 g/L of the multispecific protein. In one embodiment, the cation exchange medium is loaded with 15 g/L to 20 g/L of the multispecific protein. In a related embodiment, the cation exchange medium is loaded with 15 g/L to 35 g/L of the multispecific protein. In a related embodiment, the cation exchange medium is loaded with 20 g/L to 35 g/L of the multispecific protein. In a related embodiment, the cation exchange medium is loaded with 20 g/L to 30 g/L of the multispecific protein. In a related embodiment, the cation exchange medium is loaded with 20 g/L to 25 g/L of the multispecific protein. In a related embodiment, the cation exchange medium is loaded with 25 g/L to 35 g/L of the multispecific protein. In a related embodiment, the cation exchange medium is loaded with 25 g/L to 30 g/L of the multispecific protein.

In one embodiment the invention provides that the cation exchange medium is loaded with 10 g/L of the multispecific protein, washed with at least one wash buffer comprising 105 mM sodium chloride and eluted in a salt gradient at 8 mM/CV.

In one embodiment the invention provides that the cation exchange medium is loaded with 15 g/L to 30 g/L of the multispecific protein and washed with at least one wash buffer comprising 147 mM sodium chloride.

In one embodiment the invention that the cation exchange medium is loaded with 25 g/L to 40 g/L of the multispecific protein, wherein at least one wash buffer and one elution buffer comprise 125 mM sodium chloride.

The bound multispecific protein is then eluted from the solid phase of the cation exchange chromatography medium. The multispecific protein may be eluted by a gradient. The gradient may be a linear or step gradient. The gradient may be a salt gradient. The gradient may be a linear salt gradient or a step salt gradient. For the salt gradient, the salt concentration (ionic strength) is varied with time during the gradient. An adequate salt concentration is required to disrupt the binding of the multispecific protein and to release it into the eluate. Examples of salts that that can be used include sodium chloride, potassium chloride, and acetate.

The cation exchange chromatography eluate can be subjected to further downstream polish chromatography purification unit operations. In one embodiment, following cation exchange chromatography, the multispecific protein of interest is applied to polish chromatography medium in flow-through mode.

Following the polish chromatography operations, the concentration of the purified multispecific protein and buffer exchange into a desired formulation buffer for bulk storage of the drug substance can be accomplished by an ultrafiltration and diafiltration operation.

Critical attributes and performance parameters of the purified multispecific protein can be measured to better inform decisions regarding performance of each step during manufacture. These critical attributes and parameters can be monitored real-time, near real-time, and/or after the fact. Key critical parameters such as media components that are consumed (such as glucose), levels of metabolic by-products (such as lactate and ammonia) that accumulate, as well as those related to cell maintenance and survival, such as dissolved oxygen content can be measured during cell culture. Critical attributes such as specific productivity, viable cell density, pH, osmolality, appearance, color, aggregation, percent yield and titer may be monitored during appropriated stages in the manufacturing process. Monitoring and measurements can be done using known techniques and commercially available equipment.

The pharmaceutical compositions (solutions, suspensions or the like), may include one or more of the following: buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives; sterile diluents such as water for injection, saline solution, preferably physiological saline, Ringer's solution, isotonic sodium chloride, fixed oils such as synthetic mono- or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic.

While the terminology used in this application is standard within the art, definitions of certain terms are provided herein to assure clarity and definiteness to the meaning of the claims. Units, prefixes, and symbols may be denoted in their SI accepted form. Numeric ranges recited herein are inclusive of the numbers defining the range and include and are supportive of each integer within the defined range. The methods and techniques described herein are generally performed according to conventional methods well known in the art and as described in various general and more specific references that are cited and discussed throughout the present specification unless otherwise indicated. See, e.g., Sambrook et al. Molecular Cloning: A Laboratory Manual, 3rd ed., Cold Spring Harbor Laboratory Press, Cold Spring Harbor, N.Y. (2001) and Ausubel et al., Current Protocols in Molecular Biology, Greene Publishing Associates (1992), and Harlow and Lane Antibodies: A Laboratory Manual Cold Spring Harbor Laboratory Press, Cold Spring Harbor, N.Y. (1990). All documents, or portions of documents, cited in this application, including but not limited to patents, patent applications, articles, books, and treatises, are hereby expressly incorporated by reference.

The present invention is not to be limited in scope by the specific embodiments described herein that are intended as single illustrations of individual aspects of the invention, and functionally equivalent methods and components are within the scope of the invention. What is described in an embodiment of the invention can be combined with other embodiments of the invention. Indeed, various modifications of the invention, in addition to those shown and described herein will become apparent to those skilled in the art from the foregoing description and accompanying drawings. Such modifications are intended to fall within the scope of the appended claims.

The following examples, including the experiments conducted and the results achieved, are provided for illustrative purposes only and are not to be construed as limiting the scope of the appended claims.

### EXAMPLES

### Example 1 Single No Salt Wash Bi-Specific #1

Neutralized Protein A pool containing a fully human bi-specific, engineered immunoglobulin (Bi-specific #1) in an acetate buffer was loaded onto a Capto-SP ImpRes® cation exchange chromatography resin (GE Healthcare Bio-Science, Marlborough, Mass.) under the conditions outlined in Table 1.

TABLE 1
Conditions for cation exchange chromatography
using low salt wash

| | Bi-specific #1 |
|---|---|
| Column Size and | 20 ± 2 cm 180 cm/hr ± 10% |
| rate | |
| Concentration g/L | 20 |
| Pre-Equilibration | 500 mM Acetate 1.75M Sodium Chloride pH 4.9 |
| Equilibration | 100 mM Acetate pH 5.0 |
| Wash 1 | 100 mM Acetate pH 5.0 CV 2.0 |
| Elution Buffer A | 100 mM Acetate pH 5.0 |
| Elution Buffer B | 100 mM acetate 500 mM Sodium Chloride pH 5.0 |
| Elution Gradient | 0 |
| Start, % B | |
| Elution Gradient | 100 |
| End, % B | |
| Elution Gradient | 31.25 |
| Length, CV | |
| Elution Salt | 16.0 |
| Gradient, mM/CV | |
| Column Volumes | 17.5 |
| CVs | |
| Yield | 70% |
| Impurities in the | The entire elution step was collected by fractions. |
| recovered eluate | The best fractions resulted in 0.9% HMW, 0% NCG |

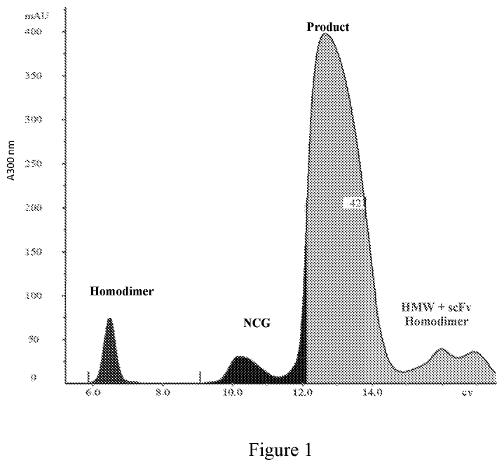

FIG. 1 shows three peaks in the Elution profile, indicating that multiple impurities remained on the column and were eluted with the main product. These impurities included homodimer, NCG (non-consensus glycosylation), and high molecular weight species (HMW).

### Example 2 High Salt Washes Bi-Specific #1

Neutralized virus inactivated pool containing Bi-specific #1 in an acetate buffer was loaded onto a Capto-SP ImpRes® cation exchange chromatography resin under the conditions described in Table 2.

TABLE 2
Conditions for cation exchange chromatography
using high salt washes

| | Bi-specific #1 |
|---|---|
| Column Size and | 20 ± 2 cm 180 cm/hr linear velocity |
| rate | |
| Concentration | 30 g/L |
| Pre Equilibration | 500 mM Acetate 1.75M Sodium Chloride pH 4.9 |
| Equilibration | 100 mM Acetate pH 5.0 |
| Wash 1 | 100 mM Acetate pH 5.0 |
| Wash 2 | 100 mM Acetate 147 mM Sodium Chloride pH 5.0 |
| | CV 2.5 |
| Wash 3 | 100 mM Acetate 70 mM Sodium Chloride pH 5.0 |
| Elution Buffer A | 100 mM Acetate pH 5.00 |
| Elution Buffer B | 100 mM Acetate 350 mM Sodium Chloride pH 5.0 |
| Elution Gradient | 20 |
| Start, % B | |
| Elution Gradient | 100 |
| End, % B | |
| Elution Gradient | 17.5 |
| Length, CV | |
| Elution Salt | 16.0 |
| Gradient, mM/CV | |
| Column Volumes | 17.5 |
| CVs | |
| Yield | 70% |
| Impurities in the | 1.6% HMW |
| recovered eluate | 0.5% NCG |

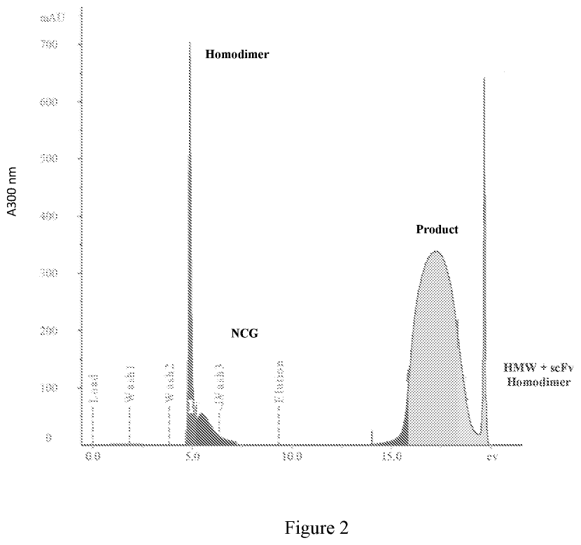

It was found that when a high salt wash was added, the lower pI impurities that were previously removed in the elution eluate, were now removed from the resin in the wash before elution. FIG. 2 shows that addition of the high salt wash resulted in a reduction in the number of impurity peaks in the elution profile, from three peaks to a single peak. The majority of the impurities were removed between the second and third wash steps, ≥68% of the NCG and ≥80% of the homodimer. The homodimers and NCG were predominantly removed from the resin during the second wash or minimally bound to the resin. The third wash step reestablished the UV baseline to zero before the start of the elution, tightening the elution profile, resulting in a much more efficient collection and better quality of the main product. Addition of the high salt wash step optimized the purification making it a more robust and manufacturing friendly process that reduced out of spec results for the product quality and maintained an acceptable yield.

These conditions were effective with load concentrations of 15-30 g/L of Bi-specific #1.

In addition, 0.05 pH units above and below pH 5.0 of the wash buffers (pH 4.95-5.05) were tested and found to be effective for the removal of low pI product-related impurities, consistent with the results above.

It was found that using 2.5 column volumes of the second wash buffer was sufficient to wash/elute the product-related impurities off the cation exchange medium. Fewer column volumes were not as efficient at removing the product-related impurities and using greater than 2.5 column volumes began to elute the main product.

### Example 3 Single No Salt Wash Bi-Specific #2

Neutralized Protein A eluate pool (100 mM Acetate, pH 5.0) containing an engineered fully human anti-hetero-IgG bispecific antibody (Bi-specific #2), was loaded onto a Capto-SP ImpREs® cation exchange chromatography resin (GE Healthcare Bio-Science, Marlborough, Mass.) under the conditions outlined in Table 3.

TABLE 3
Conditions for cation exchange chromatography
under low salt wash conditions

| | Bi-specific #2 |
|---|---|
| Column Size and | 4.66 mL, 140% linear velocity (cm/hr) |
| rate | |
| Concentration | 25 |
| Loading level (g/L) | |
| Pre Equilibration | 500 mM Acetate 1.75M Sodium Chloride pH 4.9 |
| Equilibration | 100 mM Acetate pH 5.0 |
| Wash | 100 mM Acetate pH 5.0 |
| Elution Buffer A | 100 mM Acetate pH 5.0 |
| Elution Buffer B | 100 mM Acetate 500 mM Sodium Chloride pH 5.0 |
| Elution Gradient | 0 |
| Start, % B | |
| Elution Gradient | 100 |
| End, % B | |
| Elution Gradient | 31 |
| Length, CV | |
| Elution Salt | 16 |
| Gradient, mM/CV | |
| Pool Column | 2.5 |
| Volumes | |
| Yield (from pooled | 65% |
| fractions 7 to 11) | |
| Impurities in the | SE-HPLC HMW (%) = 1.5% |
| recovered eluate | SE-HPLC LMW (%) = 0.9% |
| (from pooled | Half antibodies = 0% |
| fractions 7 to 11) | Reduced CE/SDS LC1/LC2 ratio = 1.40 |

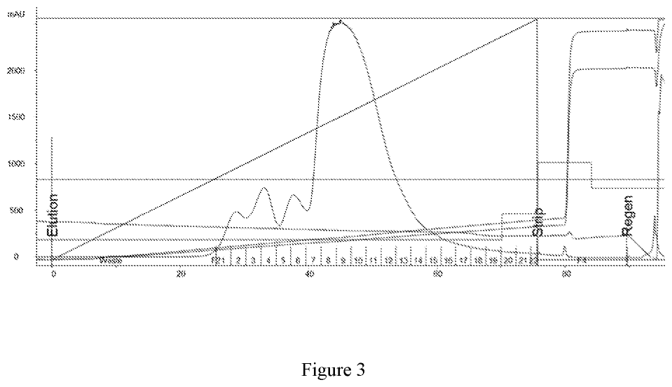

FIG. 3 shows that multiple impurities remained on the column and were eluted with the main product. These impurities included half antibodies (fractions 1, 2, 3, 4 with 50% half antibodies), 2× light chain-mis-assemblies (Fractions 5 and 6 with a LC1 to LC2 ratio <0.4 indicating LC2 assembled incorrectly with HC1), and high molecular weight (HMW, fractions 12 to 21). While the resolution of the chromatographic separation was acceptable with distinct pre-peaks containing the impurities, the manufacturing process made use of an automatic pooling based on OD; for which it would be necessary to collect the eluate by starting above the highest OD for the pre-peaks, lowering the yield, and making this process insufficient for use in a manufacturing operation.

### Example 4 High Salt Wash Bi-Specific #2

Neutralized Protein A eluate pool (100 mM Acetate, pH 5.0) containing Bi-specific #2, was loaded onto a Capto-SP ImpREs® cation exchange chromatography resin (GE Healthcare Bio-Science, Marlborough, Mass.) under the conditions outlined in Table 4.

TABLE 4
Conditions for cation exchange chromatography
under high salt wash conditions

| | Bi-specific #2 |
|---|---|
| Column Size and | 4.66 mL, 140% linear velocity (cm/hr) |
| rate | |
| Concentration | 20 |
| Loading level (g/L) | |
| Pre Equilibration | 500 mM Acetate 1.75M Sodium Chloride pH 4.9 |
| Equilibration | 100 mM Acetate pH 5.0 |
| Wash 1 | 100 mM Acetate pH 5.0 |
| Wash 2 | 100 mM Acetate 100 mM Sodium Chloride pH 5.0 |
| Wash 3 | 100 mM Acetate pH 5.0 |
| Elution Buffer A | 100 mM Acetate pH 5.0 |
| Elution Buffer B | 100 mM Acetate 500 mM Sodium Chloride pH 5.0 |
| Elution Gradient | 0 |
| Start, % B | |
| Elution Gradient | 100 |
| End, % B | |
| Elution Gradient | 31 |
| Length, CV | |
| Elution Salt | 16.0 |
| Gradient, mM/CV | |
| Pool Column | 3 |
| Volumes | |
| Yield (from pooled | 73% |
| fractions 5-10) | |
| Impurities in the | SE-HPLC HMW (%) = 1.4 |
| recovered eluate | SE-HPLC LMW (%) = 0.6 |
| (corresponding to | Half antibodies = 0.4% |
| Fractions 5-10) | Reduced CD/SDS LC1/LC2 ratio = 0.8 |

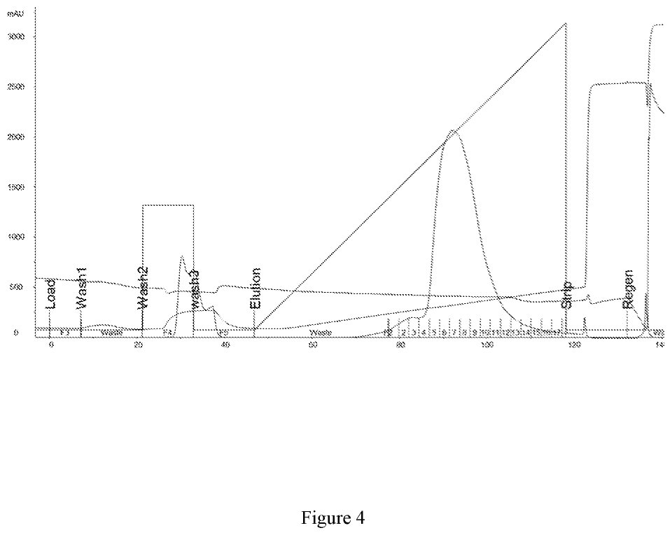

It was found that when a high salt wash step was added, (wash 2 with 100 mM sodium chloride), the half antibodies impurities were removed from the CEX resin before elution (100% half antibodies were detected in collected wash 2 and 3). FIG. 4 shows that the high salt wash resulted in a reduction in the number of peaks in the elution profile, from four peaks to a single peak with a small shoulder that still contained 2×LC2 mispaired species (LC1/LC2<0.11 as compared to the expected ratio of 1 when LC1 and LC2 assemble correctly to HC1 and HC2, respectively). The third wash step also reestablished the UV baseline to zero before the start of the elution, tightening the elution profile, resulting in a much more efficient collection and better quality of the main product. This optimized procedure with a higher salt wash, is suitable for use on the manufacturing floor. CEX purification yield increased from 65% to 73%, with an adequate elution profile for collecting a purified pool with low levels of half antibodies, mispaired LC2 species (as evidence by the LC1 to LC2 ratio close to 1), HMW and LMW, and an absorbance-based pooling criterion.

### Example 5 Single No Salt Wash Bi-Specific #3

Neutralized Protein A eluate pool containing a fully human, engineered IgG/Fab fusion protein (Bi-specific #3), was loaded onto a Capto-SP ImpREs® cation exchange chromatography resin (GE Healthcare Bio-Science, Marlborough, Mass.) under the conditions outlined in Table 5.

TABLE 5
Conditions for high load density cation exchange
chromatography with no salt load conditioning

| | Bi-specific #3 |
|---|---|
| Column Size and | 4.66 mL |
| rate | 140 cm/h linear velocity |
| Concentration | 25 |
| Loading level | |
| (mg/ml) | |
| Pre Equilibration | 500 mM Acetate 1.75M Sodium Chloride pH 4.9 |
| Equilibration | 100 mM Acetate pH 5.0 |
| Wash | 100 mM Acetate pH 5.0 |
| Elution Buffer A | 100 mM Acetate pH 5.0 |
| Elution Buffer B | 100 mM Acetate 500 mM Sodium Chloride pH 5.0 |
| Elution Gradient | 0 |
| Start, % B | |
| Elution Gradient | 100 |
| End, % B | |
| Elution Gradient | 31 |
| Length, CV | |
| Elution Salt | 16 |
| Gradient, mM/CV | |
| Pool Column | 1.5 |
| Volumes | |
| Yield | 44% |
| Impurities in the | Reduced CR/SDS LC1/LC2 ratio = 0.9 |
| recovered eluate | SE-HPLC HMW (%) =1.7 |
| (corresponding to | SE-HPLC LMW (%) = 0.9 |
| Fractions 3, 5, | SE-HPLC Monomer = 97.4% |
| and 6) | |

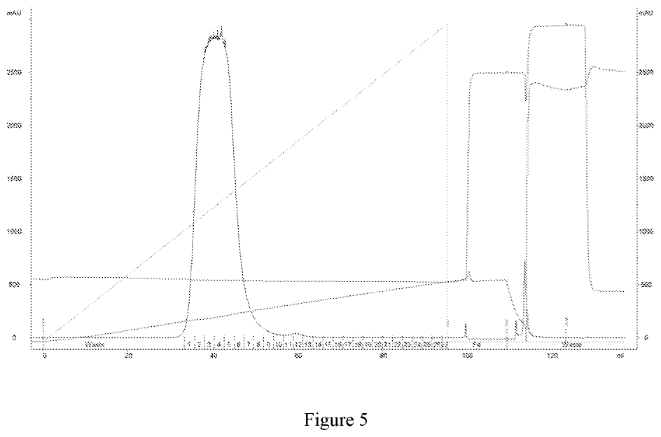

FIG. 5 shows one elution peak resulting from the high load density at a steep elution gradient. The low pI product-related impurities did not resolve from the main product under the high load density and are mostly in fractions 1, 2, and 3 as shown by a reduced CE-SDS LC1 to LC2 ratios of 4 to 7 (mispaired LC species) and LMW species of 2 to 4%.

### Example 6 Lower Load Density, Single No Salt Wash, Bi-Specific #3

The neutralized Protein A eluate pool containing the fully human, engineered IgG/Fab fusion protein (Bi-specific #3), was loaded onto a Capto-SP ImpREs® cation exchange chromatography resin (GE Healthcare Bio-Science, Marlborough, Mass.) under the conditions outlined in Table 6.

TABLE 6
Conditions for lower load density cation exchange
chromatography under no salt load conditions

| | Bi-specific #3 |
|---|---|
| Column Size and | 4.66 mL |
| rate | 140 cm/h linear velocity |
| Concentration | 10 |
| Loading level | |
| (mg/mL) | |
| Pre Equilibration | 500 mM Acetate 1.75M Sodium Chloride pH 4.9 |
| Equilibration | 100 mM Acetate pH 5.0 |
| Wash | 100 mM Acetate pH 5.0 |
| Elution Buffer A | 100 mM Acetate pH 5.0 |
| Elution Buffer B | 100 mM Acetate 500 mM Sodium Chloride pH 5.0 |
| Elution Gradient | 0 |
| Start, % B | |
| Elution Gradient | 100 |
| End, % B | |
| Elution Gradient | 62 |
| Length, CV | |
| Elution Salt | 8 |
| Gradient, mM/CV | |
| Pool Column | 3.0 |
| Volumes | |
| Yield | 73% |
| Impurities in the | Reduced CE-SDS LC1/LC2 ratio = 1.2 |
| recovered eluate | SE-HLPC HMW (%) = 1.4 |
| (corresponding to | SE-HPLC LMW (%) = 0.1 |
| fractions 5, 6, 7, | SE-HPLC Monomer = 98.4% |
| 8, 9, and 10) | |

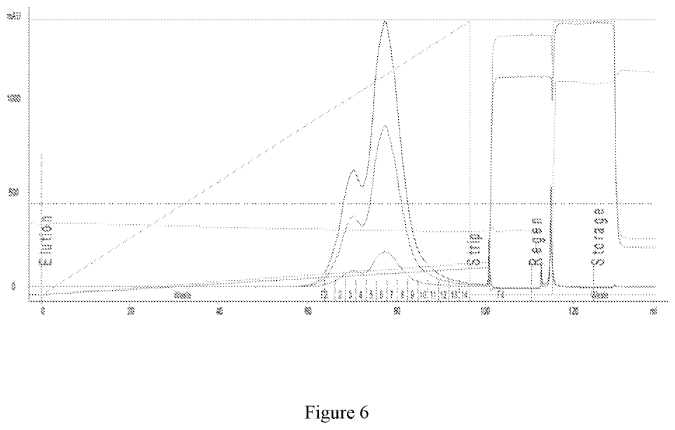

Since the high load density, steep elution gradient conditions of Example 5 did not provide sufficient resolution of the main product from the low pc product-related impurities, the load density and gradient conditions were reduced. FIG. 6 shows that a lower loading density (10 vs 25 g/L) and shallower gradient (8 vs 16 mM/CV) allowed for separation of the main low p product-impurities into a distinct peak formed by fractions 1 to 4. This fraction showed a LC1 to LC2 ratio of 3 to 10, indicating mispaired LC1 species. In contrast, the main peak showed a cumulative LC1 to LC2 ratio of 1.2. While the resolution was better and increased the yield from 44% to 73%, because it still required automatic pooling based on GD, it would still be necessary to collect the eluate by started above the highest GD for the pre-peak, lowering the yield, making this process insufficient for use in a manufacturing operation.

### Example 7 High Salt Wash, Bi-Specific #3

Neutralized virus inactivated pool containing Bi-specific #3 was loaded onto a Capto-SP ImpREs® cation exchange chromatography resin (GE Healthcare Bio-Science, Marlborough, Mass.) under the conditions outlined in Table 7.

TABLE 7
Conditions for cation exchange chromatography
under high salt load.

| | Bi-specific #3 |
|---|---|
| Column Size and | 4.66 mL |
| rate | 140 cm/h linear velocity |
| Concentration g/L | 10 |
| Pre Equilibration | 500 mM Acetate 1.75M Sodium Chloride pH 4.9 |
| Equilibration | 100 mM Acetate pH 5.0 |
| Wash 1 | 100 mM Acetate pH 5.0 |
| Wash 2 | 100 mM Acetate 105 mM Sodium Chloride pH 5.0 |
| Wash 3 | 100 mM Acetate pH 5.0 |
| Elution Buffer A | 100 mM Acetate pH 5.0 |
| Elution Buffer B | 100 mM Acetate 500 mM Sodium Chloride pH 5.0 |
| Elution Gradient | 0 |
| Start, % B | |
| Elution Gradient | 100 |
| End, % B | |
| Elution Gradient | 62 |
| Length, CV | |
| Elution Salt | 8.1 |
| Gradient, mM/CV | |
| Pool Column | |
| Volumes | 3.0 |
| Yield | 66 |
| Impurities in the | Reduced CE-SDS LC1 to LC2 ratio =1.1 |
| recovered eluate | |
| (corresponding to | SE-HPLC HMW = 1.4% |
| fractions 7, 8, 9, | SE HPLC LMW = 0.0 |
| 10, 11, and 12) | SE-HPLC Monomer = 98.6% |

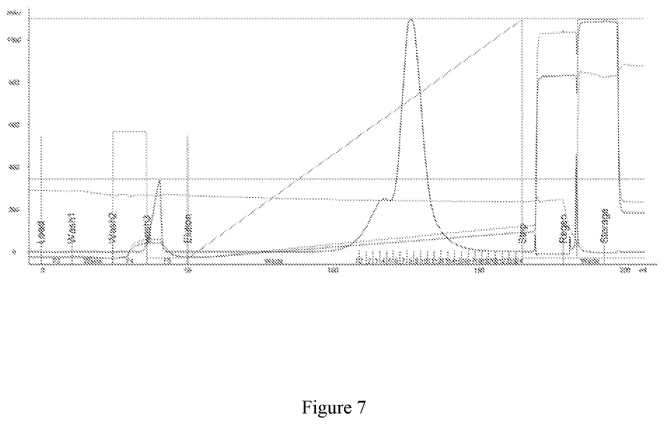

It was found that when a high salt wash step was added, (Wash 2), the impurities were removed from the CEX resin before elution. These impurities likely corresponded to mispaired LC1 species given that the LC1 to LC2 ratio on the collected wash 2 and wash 3 was 8.0, as compared to the expected ratio of 1 when LC1 and LC2 correctly assemble and pair. FIG. 7 shows that the high salt wash resulted in a reduction in the number of impurity peaks in the elution profile, from two peaks to a single peak with a small shoulder that still contained mispaired species (LC1/LC2=2 to 4). The majority of the impurities were removed during the second and third wash steps. The third wash step also reestablished the UV baseline to zero before the start of the elution, tightening the elution profile, resulting in a much more efficient collection and better quality of the main product. This optimized procedure with a higher salt wash, combined with a lower load level and shallower gradient, is suitable for use on the manufacturing floor. CEX purification yield increased from 44% to 66%, with an elution profile for collecting a purified pool with low levels of mispaired LC1 species (as evidence by the LC1 to LC2 ratio close to 1), HMW and LMW, and an absorbance-based pooling criteria.

### Example 8 High Salt Wash Bi-Specific #4

Neutralized low pH viral inactivated Protein A eluate pool containing a fully human engineered immunoglobulin bi-specific antibody (Bi-specific #4), was loaded onto a Capto-SP ImpREs® cation exchange chromatography resin under the conditions described in Table 7.

TABLE 7
Conditions for cation exchange chromatography
under high salt wash conditions

| | Bi-specific #4 |
|---|---|
| Column rate | 20 cm, 180 cm/h linear velocity |
| Concentration g/L | 35 |
| Pre Equilibration | 200 mM Acetate 1.0M Sodium Chloride pH 5.0 |
| Equilibration | 100 mM Acetate pH 5.0 |
| Wash 1 | 100 mM Acetate pH 5.0 |
| Wash 2 | 100 mM Acetate 125 mM Sodium Chloride pH 5.0 |
| Elution Buffer A | 100 mM Acetate 125 mM Sodium Chloride pH 5.0 |
| Elution Buffer B | 100 mM Acetate 500 mM Sodium Chloride pH 5.0 |
| Elution Gradient | 0 |
| Start, % B | |
| Elution Gradient | 100 |
| End, % B | |
| Elution Gradient | 10 |
| Length, CV | |
| Elution Salt | 37.5 |
| Gradient, mM/CV | |
| Average Pool | 2.5 |
| Column Volumes | 2-3 (second run) |
| (CV) | |
| Yield | 66% |
| | 77.7% (second run) |
| Impurities in the | SE-HPLC HMW = 1.3% (second run 1.2) |
| recovered eluate | SE HPLC LMW = 0.6 (second run 0.5) |
| | % Main Peak = 98.1% (second run 98.3) |
| | % Post Peak = 0.0 for both runs |
| | % Pre Peaks = 0.0 for both runs |

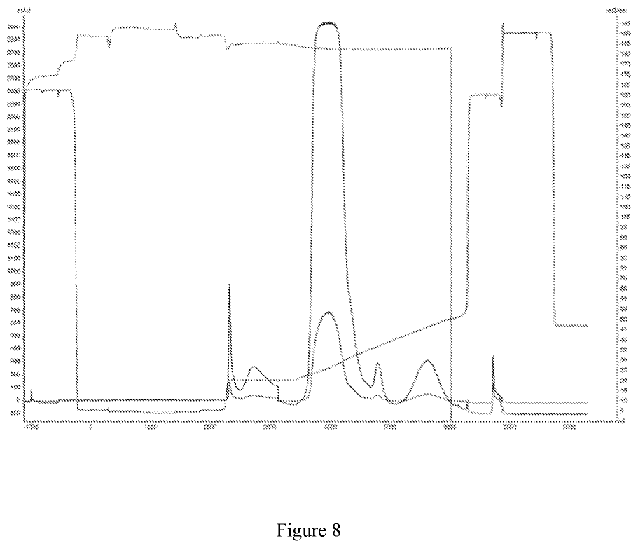

It was found that when a wash step at a high salt concentration was added (Wash 2), the low pI product-related impurities (homodimers and aggregated species) were removed from the cation exchange medium prior to elution. FIG. 8 shows that the high salt wash resulted in a reduction in the number of impurity peaks in the elution profile to a single peak. A first wash without sodium chloride brought the conductivity to base line. Low pI product-related impurities were removed during the following high salt wash step and returned the conductivity baseline to zero prior to elution. The conductivity was maintained with Elution Buffer A, which had the same high salt formulation as the high salt wash buffer. This allowed for maintaining stable conductivity before elution started and tightening the elution profile, resulting in a much more robust and efficient collection and better quality of the main product, since the separation was based on pI.

In addition, 0.1 pH units above and below pH 5.0 of the wash buffers (pH 4.9-5.1) were tested and found to be equally effective for the removal of low pI product-related impurities.

In addition, load concentration from 25 to 40 g/L-r of Bi-specific #4 were tested and found to have similar impurity clearance as the 35 g/L-r condition described above.

What is claimed is:

1. A method for purifying a multispecific protein comprising
loading a sample comprising a multispecific protein onto a cation exchange chromatography medium;
washing the cation exchange medium with at least one wash buffer comprising 100-147 mM sodium chloride; and
eluting the multispecific protein from the cation exchange chromatography resin.

2. The method according to claim 1, wherein at least one wash buffer comprises 100-125 mM sodium chloride.

3. The method according to claim 1, wherein at least one wash buffer comprises 100-105 mM sodium chloride.

4. The method according to claim 1, wherein at least one wash buffer comprises 105-147 mM sodium chloride.

5. The method according to claim 1, wherein at least one wash buffer comprises 105-125 mM sodium chloride.

6. The method according to claim 1, wherein at least one wash buffer comprises 125-147 mM sodium chloride.

7. The method according to claim 1, wherein at least one wash buffer comprises acetate.

8. The method according to claim 7, wherein at least one wash buffer comprises acetate, pH 5.0±0.05% to pH 5.0±0.1%.

9. The method according to claim 7, wherein at least one wash buffer comprises acetate, pH 4.91-5.1.

10. The method according to claim 7, wherein at least one wash buffer comprises acetate, pH 4.9, 5.0, or 5.1.

11. The method according to claim 7, wherein the wash buffer comprises 100 mM acetate.

12. The method according to claim 1, wherein at least one wash buffer comprises acetate, 100-125 mM sodium chloride.

13. The method according to claim 1, wherein at least one wash buffer comprises acetate, 100-105 mM sodium chloride.

14. The method according to claim 1, wherein at least one wash buffer comprises acetate, 105-147 mM sodium chloride.

15. The method according to claim 1, wherein at least one wash buffer comprises acetate, 105-125 mM sodium chloride.

16. The method according to claim 1, wherein at least one wash buffer comprises acetate, 125-147 mM sodium chloride.

17. The method according to claim 1, wherein the cation exchange medium is washed with at least two wash buffers.

18. The method according to claim 1, wherein the cation exchange medium is washed with at least three wash buffers.

19. The method according to claim 18, wherein the cation exchange medium is washed with at least two wash buffers, at least one of the wash buffers comprising 0-147 mM sodium chloride.

20. The method according to claim 19, wherein the cation exchange medium is washed with at least two wash buffers, at least one of the wash buffers comprising 0-70 mM sodium chloride.

21. The method according to claim 1 wherein the cation exchange medium is washed with at least two wash buffers, at least one wash buffer comprising acetate, 0 mM sodium chloride, followed by a wash buffer comprising acetate, 100-147 mM sodium chloride.

22. The method according to claim 21 wherein the cation exchange medium is washed with a wash buffer comprising acetate, 0 mM sodium chloride, followed by a wash buffer selected from the group consisting of a wash buffer comprising acetate, 100 mM sodium chloride, a wash buffer comprising acetate, 105 mM sodium chloride, or a wash buffer comprising acetate, 125 mM sodium chloride.

23. The method according to claim 21, wherein the cation exchange medium is washed with a wash buffer comprising acetate, 100-147 mM sodium chloride, followed by a wash buffer comprising acetate, 0-70 mM sodium chloride.

24. The method according to claim 23, wherein the cation exchange medium is washed with a wash buffer comprising acetate, 100-147 mM sodium chloride, followed by a wash buffer comprising acetate, 0 mM sodium chloride.

25. The method according to claim 23, wherein the cation exchange medium is washed a wash buffer comprising acetate, 100-147 mM sodium chloride, followed by a wash buffer comprising 70 mM sodium chloride.

26. The method according to claim 18, wherein the cation exchange medium is washed with at least three wash buffers,
a first wash buffer comprising acetate, 0 mM sodium chloride, followed by
a second wash buffer comprising acetate, 100-147 mM sodium chloride, followed by
a third wash buffer comprising acetate, 0 mM sodium chloride or a wash buffer comprising acetate, 70 mM sodium chloride.

27. The method according to claim 26, wherein the cation exchange medium is washed with
a first wash buffer comprising acetate, 0 mM sodium chloride, followed by
a second wash buffer selected from the group consisting of a wash buffer comprising acetate, 100 mM sodium chloride, a wash buffer comprising acetate, 105 mM sodium chloride, or a wash buffer comprising acetate, 125 mM sodium chloride, followed by
a third a wash buffer comprising acetate, 0 mM sodium chloride.

28. The method according to claim 26, wherein the cation exchange medium is washed with
a first wash buffer comprising acetate, 0 mM sodium chloride, followed by
a second wash buffer comprising acetate, 147 mM sodium chloride, followed by
a third a wash buffer comprising acetate, 70 mM sodium chloride.

29. The method according to claim 1, wherein the cation exchange medium is washed with 2.5 mM/CV of a wash buffer comprising 147 mM sodium chloride.

30. The method according to claim 1, wherein the multispecific protein is eluted from the cation exchange medium by a gradient.

31. The method according to claim 30, wherein the gradient is a linear or step gradient.

32. The method according to claim 30, wherein the gradient is a salt gradient.

33. The method according to claim 30, wherein at least one of the buffers used to form the elution gradient comprises 0-1M sodium chloride.

34. The method according to claim 33, wherein at least one of the buffers used to form the elution gradient comprises 70-500 mM sodium chloride.

35. The method according to claim 33, wherein at least one of the buffers used to form the elution gradient comprises 125 mM sodium chloride.

36. The method according to claim 1, wherein at least one wash buffer and one elution buffer comprise 125 mM sodium chloride.

37. The method according to claim 1, wherein the cation exchange medium is loaded with at least 10 g/L of the multispecific protein.

38. The method according to claim 1, wherein the cation exchange medium is loaded with 10 g/L to 40 g/L of the multispecific protein.

39. The method according to claim 38, wherein the cation exchange medium is loaded with 15 g/L to 30 g/L.

40. The method according to claim 38, wherein the cation exchange medium is loaded with 25 g/L-40 g/L.

41. The method according to claim 1, wherein the cation exchange medium is loaded with 10 g/L of the multispecific protein, washed with a wash buffer comprising 105 mM sodium chloride and eluted in a salt gradient at 8 mM/CV.

42. The method according to claim 1, wherein the cation exchange medium is loaded with 15 g/L to 30 g/L of the multispecific protein, washed with a wash buffer comprising 147 mM sodium chloride.

43. The method according to claim 1, wherein the cation exchange medium is loaded with 25 g/L to 40 g/L of the multispecific protein, wherein at least one wash buffer and one elution buffer comprise 125 mM sodium chloride.

44. The method according to claim 1, wherein at least one product-related impurity is a homodimer, high molecular weight species, half antibody, aggregate, low molecular weight species, antibody fragment, or a light chain mis-assembly.

45. A purification process that includes a unit operation comprising cation exchange chromatography preformed according to the method of claim 1.

46. The method according to claim 1, further comprising, before and/or after the cation exchange chromatography step, one or more unit operations for purifying the multispecific protein, comprising affinity chromatography, ion exchange chromatography, hydrophobic interaction chromatography column, and/or mixed-mode chromatography column.

47. The method according to claim 1, wherein the multispecific protein is a bispecific protein.

48. The method according to claim 1, wherein the multispecific protein is a bispecific antibody.

49. A purified, multispecific protein produced according to the method of claim 1.

50. The method according to claim 1, wherein the cation exchange chromatography medium is a resin.

51. A method for reducing low pI product-related impurities in the eluate from cation exchange chromatography, the method comprising
loading a composition comprising a multispecific protein and at least one product-related impurity having a pI lower than the multispecific protein, onto a cation exchange chromatography medium;
washing the cation exchange medium with a first wash buffer,
washing the cation exchange medium with a second wash buffer comprises 100-147 mM sodium chloride;
eluting the multispecific protein from the cation exchange chromatography resin;
wherein the cation exchange chromatography eluate has reduced low pI product-related impurities compared to the cation exchange chromatography eluate recovered in a corresponding method in which no sodium chloride is included in a wash buffer formulation.

52. The method according to claim 51, wherein the cation exchange medium is washed with a third wash buffer.

53. A method for performing cation exchange chromatography under high salt wash conditions to reduce product-related impurities, the method comprising
loading a composition comprising a multispecific protein and at least one product-related impurity onto an equilibrated cation exchange column;
washing the cation exchange medium with at least two wash buffers, wherein one wash buffer comprising 100-147 mM sodium chloride; and
eluting the bound multispecific protein from the cation exchange chromatography resin.

54. The method according to claim 53, wherein prior to loading the composition, the cation exchange medium is equilibrated with a buffer that contains no sodium chloride.

55. A method for producing an isolated, purified, recombinant multispecific protein, the method comprising
establishing a cell culture in a bioreactor with a host cell expressing the multispecific protein;
culturing the host cells to express the multispecific protein;
harvesting the recombinant multispecific protein from the cell culture;
affinity purifying the recombinant multispecific protein;
loading the affinity purified recombinant multispecific protein onto a cation exchange chromatography resin;
washing the cation exchange resin with at least one wash comprising 100-147 mM sodium chloride;
eluting the multispecific protein from the cation exchange chromatography resin; and
loading the cation exchange chromatography eluate comprising the recombinant multispecific protein onto an additional chromatography medium in flow through mode.

56. The method according to claim 55, wherein the additional chromatography medium is selected from cation exchange chromatography medium, multi-modal chromatography medium, hydrophobic interaction chromatography medium, and hydroxyapatite chromatography medium.

57. The method according to claim 55, wherein the affinity purified multispecific protein is in an eluate pool and is subjected to low pH viral inactivation, followed by neutralization, prior to loading onto the cation exchange medium.

58. The method according to claim 56, wherein the flow through from the third chromatography medium is subjected to an ultrafiltration and diafiltration unit operation.

59. An isolated, purified, recombinant multispecific protein according to the method of claim 55.

60. A pharmaceutical composition comprising the isolated, purified, recombinant multispecific protein produced with the method of claim 55.
